SEQUENCE LISTING (52 sequences)

SEQ ID NO: 1 - DNA, 51 bp, Artificial Sequence
  Description of Artificial Sequence Synthetic 7NN_P target oligonucleotide
tggcatacaagttttcaaanngtcgtacgacnntttgacaatcgtctgtca

SEQ ID NO: 2 - DNA, 26 bp, Artificial Sequence
  Description of Artificial Sequence Synthetic Gal10F primer
gcaactttagtgctgacacatacagg

SEQ ID NO: 3 - DNA, 48 bp, Artificial Sequence
  Description of Artificial Sequence Synthetic Ulib7NNRev oligonucleotide
atgtttaaacttataagactggtttggmbnaatmbnagcgatgatgct

SEQ ID NO: 4 - DNA, 24 bp, Artificial Sequence
  Description of Artificial Sequence Synthetic Gal10R primer
acaaccttgattggagacttgacc

SEQ ID NO: 5 - DNA, 45 bp, Artificial Sequence
  Description of Artificial Sequence Synthetic Ulib7NNFor oligonucleotide
tcttataagtttaaacatcagctaagcttgnvktttcaggtgact

SEQ ID NO: 6 - DNA, 51 bp, Artificial Sequence
  Description of Artificial Sequence Synthetic 5NNC_P target oligonucleotide
tggcatacaagttttcaaaacnncgtacgnngttttgacaatcgtctgtca

SEQ ID NO: 7 - DNA, 60 bp, Artificial Sequence
  Description of Artificial Sequence Synthetic Cre44Rev primer
cactagtttgtccagaaaccaacggcgctgggtcttttgagtcacmnnaaaggtcaagct

SEQ ID NO: 8 - DNA, 57 bp, Artificial Sequence
  Description of Artificial Sequence Synthetic Cre68For primer
tttctggacaaactagtggatgaaattggcgttggttacgtannkgatcgcggatcc

SEQ ID NO: 9 - DNA, 27 bp, Artificial Sequence
  Description of Artificial Sequence Synthetic Cre43Rev primer
aaaggtcaagcttagctgatgtttaaa

SEQ ID NO: 10 - DNA, 39 bp, Artificial Sequence
  Description of Artificial Sequence Synthetic Cre44For primer
catcagctaagcttgacctttnnkgtgactcaaaagacc

SEQ ID NO: 11 - DNA, 27 bp, Artificial Sequence
  Description of Artificial Sequence Synthetic Cre67Rev primer
tacgtaaccaacgccaatttcatccac

SEQ ID NO: 12 - DNA, 57 bp, Artificial Sequence
  Description of Artificial Sequence Synthetic Cre68-77For primer
ggcgttggttacgtannkgatcgcggatccgtttccgattacnnkttaagcgaaatc

SEQ ID NO: 13 - DNA, 24 bp, Artificial Sequence
  Description of Artificial Sequence Synthetic Comb75assFor oligonucleotide
tttnnngtgactcaaaagacccag

SEQ ID NO: 14 - DNA, 24 bp, Artificial Sequence
  Description of Artificial Sequence Synthetic Comb75assRev oligonucleotide
ctgggtcttttgagtcacnnnaaa

SEQ ID NO: 15 - DNA, 51 bp, Artificial Sequence
  Description of Artificial Sequence Synthetic 7NNNN_P target oligonucleotide
tggcatacaagttttcaaannnncgtacgacnnnntgacaatcgtctgtca

SEQ ID NO: 16 - DNA, 51 bp, Artificial Sequence
  Description of Artificial Sequence Synthetic Ulib7NNForBis oligonucleotide
tcttataagtttaaacatcagctaagcttgnvktttnnkgtgactcaaaag

SEQ ID NO: 17 - DNA, 84 bp, Artificial Sequence
  Description of Artificial Sequence Synthetic CCM2For primer
aagcagagctctctggctaactagagaacccactgcttactggcttatcgaccatggccaataccaaatataacaaagagttcc

SEQ ID NO: 18 - DNA, 38 bp, Artificial Sequence
  Description of Artificial Sequence Synthetic CCMRevBis oligonucleotide
ctgctctagattagtcggccgccggggaggatttcttc

SEQ ID NO: 19 - protein, 167 aa, Artificial Sequence
  Description of Artificial Sequence Synthetic Br1 Mutant polypeptide
MANTKYNKEFLLYLAGFVDGDGSIIAAITPNQSYKFKHQLSLKFVVTQKTQRRWFLDKLVDEIGVGYVRDRGSVSDYHLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 20 - protein, 167 aa, Artificial Sequence
  Description of Artificial Sequence Synthetic Br2 Mutant polypeptide
MANTKYNKEFLLYLAGFVDGDGSIIAYISPNQSYKFKHQLSLRFVVTQKTQRRWFLDKLVDEIGVGYVLDRGSVSDYLLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 21 - protein, 167 aa, Artificial Sequence
  Description of Artificial Sequence Synthetic Mt1 Mutant polypeptide
MANTKYNKEFLLYLAGFVDGDGSIIAWIAPNQSYKFKHQLSLRFIVTQKTQRRWFLDKLVDEIGVGYVYDRGSVSDYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 22 - protein, 167 aa, Artificial Sequence
  Description of Artificial Sequence Synthetic Mt2 Mutant polypeptide
MANTKYNKEFLLYLAGFVDGDGSIIAWITPNQSYKFKHQLSLRFIVTQKTQRRWFLDKLVDEIGVGYVYDRGSVSDYLLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 23 - protein, 167 aa, Artificial Sequence
  Description of Artificial Sequence Synthetic Mt3 Mutant polypeptide
MANTKYNKEFLLYLAGFVDGDGSIIACISPNQSYKFKHQLSLRFVVTQKTQRRWFLDKLVDEIGVGYVYDRGSVSDYLLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 24 - protein, 167 aa, Artificial Sequence
  Description of Artificial Sequence Synthetic BrA Mutant polypeptide
MANTKYNKEFLLYLAGFVDGDGSIIATITPNQSYKFKHQLSLSFRVTQKTQRRWFLDKLVDEIGVGYVRDRGSVSDYKLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 25 - protein, 167 aa, Artificial Sequence
  Description of Artificial Sequence Synthetic BrB Mutant polypeptide
MANTKYNKEFLLYLAGFVDGDGSIIACISPNQSYKFKHQLSLRFRVTQKTQRRWFLDKLVDEIGVGYVSDRGSVSDYCLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 26 - protein, 167 aa, Artificial Sequence
  Description of Artificial Sequence Synthetic MtA Mutant polypeptide
MANTKYNKEFLLYLAGFVDGDGSIIASITPNQSYKFKHQLSLRFRVTQKTQRRWFLDKLVDEIGVGYVYDRGSVSDYNLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 27 - protein, 167 aa, Artificial Sequence
  Description of Artificial Sequence Synthetic MtB Mutant polypeptide
MANTKYNKEFLLYLAGFVDGDGSIIATITPNQSYKFKHQLSLRFKVTQKTQRRWLLDKLVDEIGVGYVQDRGSVSDYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 28 - protein, 167 aa, Artificial Sequence
  Description of Artificial Sequence Synthetic MtC Mutant polypeptide
MANTKYNKEFLLYLAGFVDGDGSIIATITPNQSYKFKHQLSLRFKVTQKTQRRWFLDKLVDEIGVGYVADRGSVSDYQLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 29 - DNA, 24 bp, Artificial Sequence
  Description of Artificial Sequence Synthetic 107Rev primer
agcgatgatgctaccgtcaccgtc

SEQ ID NO: 30 - DNA, 63 bp, Artificial Sequence
  Description of Artificial Sequence Synthetic SeqBrAFor primer
ggtagcatcatcgctactattactccaaaccagnvknvkaagtttaaacatnvkctaagcttg

SEQ ID NO: 31 - DNA, 63 bp, Artificial Sequence
  Description of Artificial Sequence Synthetic SeqBrBFor primer
ggtagcatcatcgcttgtatttctccaaaccagnvknvkaagtttaaacatnvkctaagcttg

SEQ ID NO: 32 - DNA, 63 bp, Artificial Sequence
  Description of Artificial Sequence Synthetic SeqBrCFor primer
ggtagcatcatcgctgctattaatccaaaccagnvknvkaagtttaaacatnvkctaagcttg

SEQ ID NO: 33 - DNA, 63 bp, Artificial Sequence
  Description of Artificial Sequence Synthetic SeqBrDFor primer
ggtagcatcatcgctgctattactccaaaccagnvknvkaagtttaaacatnvkctaagcttg

SEQ ID NO: 34 - protein, 167 aa, Artificial Sequence
  Description of Artificial Sequence Synthetic BrC Mutant polypeptide
MANTKYNKEFLLYLAGFVDGDGSIIAAINPNQSYKFKHQLSLTFQVTQKTQRRWFLDKLVDEIGVGYVRDRGSVSDYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 35 - protein, 167 aa, Artificial Sequence; identical to SEQ ID NO: 19
  Description of Artificial Sequence Synthetic BrD Mutant polypeptide

SEQ ID NO: 36 - protein, 167 aa, Artificial Sequence
  Description of Artificial Sequence Synthetic FC1 Mutant polypeptide
MANTKYNKEFLLYLAGFVDGDGSIIACISPNQTCKFKHYLSLRFRVTQKTQRRWFLDKLVDEIGVGYVSDRGSVSDYCLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 37 - protein, 167 aa, Artificial Sequence
  Description of Artificial Sequence Synthetic FC2 Mutant polypeptide
MANTKYNKEFLLYLAGFVDGDGSIIACISPNQTCKFKHCLSLRFRVTQKTQRRWFLDKLVDEIGVGYVSDRGSVSDYCLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 38 - protein, 167 aa, Artificial Sequence
  Description of Artificial Sequence Synthetic FC3 Mutant polypeptide
MANTKYNKEFLLYLAGFVDGDGSIIACISPNQGCKFKHTLSLRFRVTQKTQRRWFLDKLVDEIGVGYVSDRGSVSDYCLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 39 - protein, 167 aa, Artificial Sequence
  Description of Artificial Sequence Synthetic FC4 Mutant polypeptide
MANTKYNKEFLLYLAGFVDGDGSIIACISPNQGCKFKHHLSLRFRVTQKTQRRWFLDKLVDEIGVGYVSDRGSVSDYCLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 40 - DNA, 22 bp, Artificial Sequence
  Description of Artificial Sequence Synthetic C1234 target oligonucleotide
caaaacgtcgtgagacagtttg

SEQ ID NO: 41 - DNA, 22 bp, Artificial Sequence
  Description of Artificial Sequence Synthetic C1221_P target oligonucleotide
caaaacgtcgtacgacgttttg

SEQ ID NO: 42 - DNA, 22 bp, Artificial Sequence
  Description of Artificial Sequence Synthetic 7NN_P target oligonucleotide
caaanngtcgtacgacnntttg

SEQ ID NO: 43 - DNA, 22 bp, Artificial Sequence
  Description of Artificial Sequence Synthetic 5NN_P target oligonucleotide
caaaacnncgtacgnngttttg

SEQ ID NO: 44 - DNA, 22 bp, Artificial Sequence
  Description of Artificial Sequence Synthetic 7NNNN_P target oligonucleotide
caaannnncgtacgnnnntttg

SEQ ID NO: 45 - DNA, 22 bp, Artificial Sequence
  Description of Artificial Sequence Synthetic 7TATA_P target oligonucleotide
caaatatacgtacgtatatttg

SEQ ID NO: 46 - DNA, 22 bp, Artificial Sequence
  Description of Artificial Sequence Synthetic 7TTCT_P target oligonucleotide
caaattctcgtacgagaatttg

SEQ ID NO: 47 - DNA, 22 bp, Artificial Sequence
  Description of Artificial Sequence Synthetic 7GACT_P target oligonucleotide
caaagactcgtacgagtctttg

SEQ ID NO: 48 - DNA, 22 bp, Artificial Sequence
  Description of Artificial Sequence Synthetic 10TTG_P target oligonucleotide
cttgacgtcgtacgacgtcaag

SEQ ID NO: 49 - DNA, 22 bp, Artificial Sequence
  Description of Artificial Sequence Synthetic FullComb_P target oligonucleotide
cttggactcgtacgagtccaag

SEQ ID NO: 50 - DNA, 22 bp, Artificial Sequence
  Description of Artificial Sequence Synthetic 10NNNNNNN_P target oligonucleotide
cnnnnnnncgtacgnnnnnnng

SEQ ID NO: 51 - protein, 163 aa, Chlamydomonas reinhardtii
MNTKYNKEFLLYLAGFVDGDGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 52 - DNA, 24 bp, Artificial Sequence
  Description of Artificial Sequence Synthetic C1221_P target oligonucleotide
tcaaaacgtcgtacgacgttttga

(19) United States
(12) Patent Application Publication
(10) Pub. No.: US 2013/0149786 A1
(43) Pub. Date: Jun. 13, 2013

(54) I-CREI VARIANTS WITH NEW SPECIFICITY AND METHODS OF THEIR GENERATION

(71) Applicants: Philippe Duchateau, Livry Gargan (FR); Sylvestre Grizot, La Garenne Colombes (FR)

(72) Inventors: Philippe Duchateau, Livry Gargan (FR); Sylvestre Grizot, La Garenne Colombes (FR)

(73) Assignee: CELLECTIS, Paris (FR)

(21) Appl. No.: 13/695,551

(22) PCT Filed: Apr. 30, 2010
(86) PCT No.: PCT/IB2010/051904

§ 371 Date: Feb. 4, 2013

(51) Int. Cl. C12N 9/22; C12N 15/90

(52) U.S. Cl. CPC C12N 9/22; C12N 15/902
USPC 435/462; 435/199; 536/23.1; 435/320.1

(57) ABSTRACT

The present invention relates to 1-Cre1 variants which can in particular recognise and cleave DNA targets which do not comprise the same nucleotides at positions ±6 and ±7 which are present in the wild type 1-Cre1 target. The present invention also relates to 1-Cre1 variants which can recognise and cleave targets which do not comprise the wild type nucleotides at positions ±4, ±5, ±6, ±7 and to 1-Cre1 variants with new specificity which can recognise and cleave targets which do not comprise the wild type nucleotides at positions ±4, ±5, ±6, ±7, ±8, ±9 and ±10.

The present invention relates to I-CreI variants which can in particular recognise and cleave DNA targets which do not comprise the same nucleotides at positions ±6 and ±7 which are present in the wild type I-CreI target. The present invention also relates to I-CreI variants which can recognise and cleave targets which do not comprise the wild type nucleotides at positions ±4, ±5, ±6, ±7 and to I-CreI variants which can recognise and cleave targets which do not comprise the wild type nucleotides at positions ±4, ±5, ±6, ±7, ±8, ±9 and ±10.

Since the first gene targeting experiments in yeast more than 25 years ago (Hinnen et al, 1978; Rothstein, 1983), HR has been used to insert, replace or delete genomic sequences in a variety of cells (Thomas and Capecchi, 1987; Capecchi, 2001; Smithies, 2001). Targeted events occur at a very low frequency in mammalian cells, making the use of innate HR impractical. The frequency of HR can be significantly increased by a specific DNA double-strand break (DSB) at a locus (Rouet et al, 1994; Choulika et al, 1995). Such DSBs can be induced by meganucleases, sequence-specific endonucleases that recognize large DNA recognition target sites (12 to 30 bp).

Meganucleases show high specificity to their DNA target; these proteins can cleave a unique chromosomal sequence and therefore do not affect global genome integrity. Meganucleases are essentially represented by homing endonucleases, a widespread class of proteins found in eukaryotes, bacteria and archae (Chevalier and Stoddard, 2001). Early studies of the I-SceI and HO homing endonucleases have illustrated how the cleavage activity of these proteins can be used to initiate HR events in living cells and have demonstrated the recombinogenic properties of chromosomal DSBs (Dujon et al, 1986; Haber, 1995). Since then, meganuclease-induced HR has been successfully used for genome engineering purposes in bacteria (Posfai et al, 1999), mammalian cells (Sargent et al, 1997; Donoho et al, 1998; Cohen-Tannoudji et al, 1998), mice (Gouble et al, 2006) and plants (Puchta et al, 1996; Siebert and Puchta, 2002).

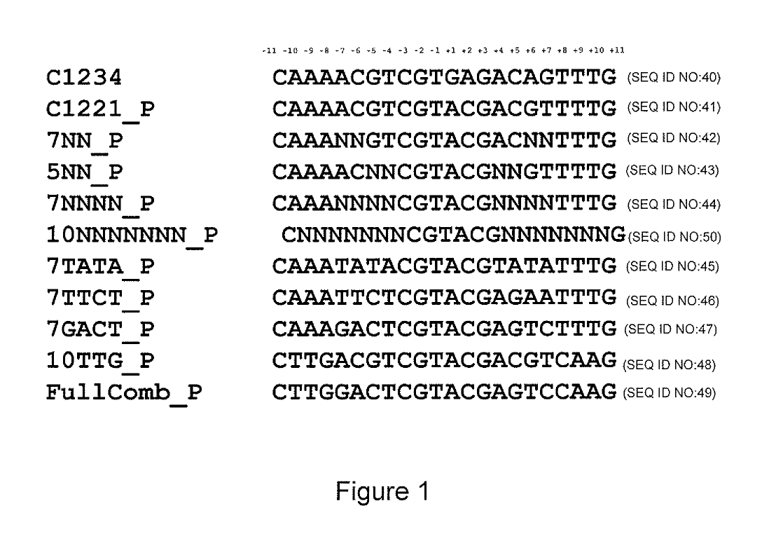

I-CreI is a meganuclease which has been studied extensively and for which the inventors and their collaborators have been able to change the I-CreI specificity toward the nucleotides at positions ±10, ±9, ±8 (10NNN region, WO2007/049156) or nucleotides at positions ±5, ±4, ±3 (5NNN region, WO2006/097853) of the wild type palindromic target of I-CreI (FIG. 1), referred to as C1221 hereafter. The inventors have also shown how these two sets of mutations can be combined in a combinatorial manner so as to generate a meganuclease which recognises and cleaves a DNA target modified at one or more nucleotides from these groups (WO2007/049095).

In previous work the specificity of I-CreI towards the 7NN nucleotides (bases at positions ±7, ±6) of the C1221 target was not modified, as a natural partial degeneracy of I-CreI exists towards this portion of the target. This 7NN degeneracy can be deduced from the wild type I-CreI C1234 target (FIG. 1), where I-CreI tolerates A or C bases at position ±7 and C or T bases at position ±6. Therefore, the four best 7NN_P targets cleaved by I-CreI are 7AC_P, 7AT_P, 7CT_P and 7CC_P.

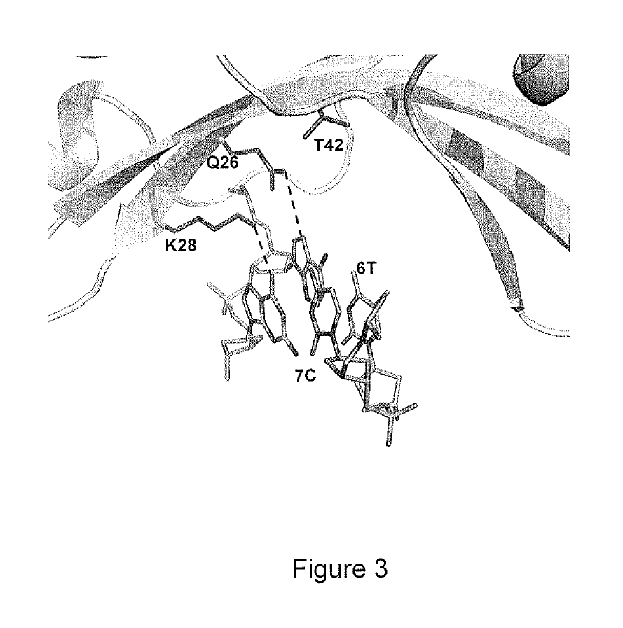

The need to generate I-CreI variants with new specificity has led the inventors to consider the possibility of changing the specificity of I-CreI toward the 7NN nucleotides by introducing substitutions at positions 26, 28 and 42 of I-CreI (FIG. 3).

For the first time the inventors have experimentally shown that it is possible to intentionally change I-CreI specificity towards the 7NN nucleotides. In particular the inventors have shown that all the 16 (4⁴) 7NN_P targets are cleaved by at least one I-CreI variant. In addition, the inventors have also shown for the first time that the 7NN nucleotides define a new DNA region able to behave as the 10NNN and 5NNN regions previously identified by the inventors, as the set of mutations conferring the new 7NN specificity can be combined in a combinatorial manner with other sets of mutations.

In order to optimize the time required for the whole engineering process (an I-CreI variant with a modified specificity toward the three 10NNN, 7NN and 5NNN DNA regions), the inventors have shown how to generate directly I-CreI variants with a modified specificity toward the nucleotides at positions ±4, ±5, ±6 and ±7 (the 256 (4⁴) palindromic 7NNNN_P targets, FIG. 1) identifying the 7NNNN nucleotides as a new DNA region. To avoid the screening against 1024 (4⁵) targets (7NNNNN_P targets), nucleotide at position ±3 was not changed.

Finally, the inventors have shown how to generate a variant directed against the complete 10NNNNNNN region, that is a target which can vary from the wild type target sequence of I-CreI at each of nucleotides ±4, ±5, ±6, ±7, ±8, ±9 and ±10, from the combination of two sets of mutations that confer new specificity toward the 7NNNN and 10NNN regions.

In the present Patent Application the terms meganuclease(s) and variant(s) and variant meganuclease(s) will be used interchangeably herein.

According to a first aspect of the present invention there is provided an I-CreI variant, having at least two substitutions, said variant being able to cleave a 7NNNN_P palindromic DNA target sequence (SEQ ID NO: 44) other than the wild type I-CreI DNA target sequence (SEQ ID NO: 40), and being obtainable by a method comprising at least the steps of:

(a) constructing a first series of I-CreI variants having at least one substitution in a position selected from the group: 26, 28, 42,

(b) constructing a second series of I-CreI variants having at least one substitution in a position selected from the group: 44, 68, 77,

(c) selecting and/or screening the variants from the first series of step (a) which are able to cleave a mutant I-CreI site wherein the nucleotides in positions ±7 to ±6 of the wild type I-CreI site have been replaced with the nucleotides which are present in positions ±7 to ±6 of said 7NNNN_P DNA target sequence,

(d) selecting and/or screening the variants from the first series of step (b) which are able to cleave a mutant I-CreI site wherein the nucleotides in positions ±5 to ±4 of the wild type I-CreI site have been replaced with the nucleotides which are present in positions ±5 to ±4 of said 7NNNN_P DNA target sequence,

(e) combining in a single variant, the mutation(s) in positions 26, 28, 42, and 44, 68, 77 of two variants from step (c) and step (d), to obtain a novel homodimeric I-CreI variant which cleaves a sequence wherein the nucleotide quartet in positions ±7 to ±4 is identical to the nucleotide quartet which is present in positions ±7 to ±4 of said 7NNNN_P DNA target sequence.

According to another aspect of the invention, homodimeric variants able to cleave a 7NNNN_P DNA target sequence can be directly generated without a combinatorial step. There is provided an I-CreI variant, having at least two substitutions, said variant being able to cleave a 7NNNN_P palindromic DNA target sequence (SEQ ID NO: 44) other than the wild type I-CreI DNA target sequence (SEQ ID NO: 40), and being obtainable by a method comprising at least the steps of:

(a′) constructing I-CreI variants having at least one substitution in a position selected from the group: 26, 28, 42, 44, 68, 77

(b′) selecting and/or screening the variants from step (a′) which are able to cleave a 7NNNN_P palindromic DNA target sequence site wherein the nucleotides in positions ±7 to ±4 of the wild type I-CreI site have been replaced with the nucleotides which are present in positions ±7 to ±4 of said 7NNNN_P DNA target sequence.

Preferably, the variants obtained in step (e) and in step (b′), also called 7NNNN cutters, are heterodimers, resulting from the association of a first and a second monomer having different mutations in positions 26, 28, 42, 44, 68, 77 of I-CreI, said heterodimers being able to cleave a non-palindromic DNA target sequence.

The inventors have now proven therefore that it is possible to create an I-CreI variant which can recognise and cleave a DNA target modified at the 7NNNN positions in a single round of selection.

According to a second aspect of the present invention there is provided an I-CreI variant, having at least two substitutions, said variant being able to cleave a 10NNNNNNN_P palindromic DNA target sequence other than the wild type I-CreI DNA target sequence (SEQ ID NO: 40), and being obtainable by a method comprising at least the steps of:

(A) selecting variants of step (c) having at least one substitution in a position selected from the group: 26, 28, 42, which are able to cleave a mutant I-CreI site wherein the nucleotides in positions ±7 to ±6 of the wild type I-CreI site have been replaced with the nucleotides which are present in positions ±7 to ±6 of said 10NNNNNNN_P DNA target sequence; or

(A′) selecting 7NNNN cutters of steps (e) and (b′) having at least two substitutions in a position selected from the group: 26, 28, 42, 44, 68, 77, which are able to cleave a mutant I-CreI site wherein the nucleotides in positions ±7 to ±4 of the wild type I-CreI site have been replaced with the nucleotides which are present in positions ±7 to ±4 of said 10NNNNNNN_P DNA target sequence,

(B) constructing a series of I-CreI variants having at least one substitution in a position selected from the group: 30, 32, 33, 38, 40,

(C) selecting and/or screening the variants from the first series of step (B) which are able to cleave a mutant I-CreI site wherein the nucleotides in positions ±10 to ±8 of the wild type I-CreI site have been replaced with the nucleotides which are present in positions ±10 to ±8 of said 10NNNNNNN_P DNA target sequence,

(D) combining in a single variant, the mutation(s) in positions 26, 28, 42, 44, 68, 77 and 30, 32, 33, 38, 40 of two variants from step (A) or (A′), and step (C), to obtain a novel homodimeric I-CreI variant which cleaves a sequence wherein the nucleotide septet in positions ±10 to ±4 is identical to the nucleotide septet which is present in positions ±10 to ±4 of said 10NNNNNNN_P DNA target sequence.

Preferably, the variant obtained in step (D) is a heterodimer, resulting from the association of a first and a second monomer having different mutations in positions 26 to 42 and 44 to 77 of I-CreI, said heterodimer being able to cleave a non-palindromic DNA target sequence.

The inventors have also shown that it is possible to generate I-CreI variants which cleavages a target which is variable across the entire 10NNNNNNN portion of the target in a simple two step process.

According to a further aspect of the present invention the variant may be obtained by a method comprising the additional steps of:

(i) constructing a third series of variants having at least one additional substitution in at least one of the monomers in said heterodimers,

(ii) combining said third series variants of step (i) and screening the resulting heterodimers for altered cleavage activity against said DNA target.

Preferably in step (i) said at least one additional substitution is introduced by site directed mutagenesis in a DNA molecule encoding said third series of variants, and/or by random mutagenesis in a DNA molecule encoding said third series of variants.

Preferably steps (i) and (ii) are repeated at least two times and wherein the heterodimers selected in step (i) of each further iteration are selected from heterodimers screened in step (ii) of the previous iteration which showed increased cleavage activity against said DNA target.

Preferably the residue at position 75 of I-CreI is not substituted.

Preferably the variant comprises one or more substitutions on the entire I-CreI sequence that improve the binding and/or the cleavage properties of the variant towards said DNA target sequence.

Preferably the substitutions are replacement of the initial amino acids with amino acids selected in the group consisting of A, D, E, F, G, H, I, K, M, N, P, Q, R, S, T, Y, C, W, L and V.

Preferably the variant is an obligate heterodimer, wherein the first and the second monomer, respectively, further comprises the D137R mutation and the R51D mutation.

Preferably the obligate heterodimer, wherein the first monomer further comprises the K7R, E8R, E61R, K96R and L97F or K7R, E8R, F54W, E61R, K96R and L97F mutations and the second monomer further comprises the K7E, F54G, L58M and K96E or K7E, F54G, K57M and K96E mutations.

Alternatively the variant consists of a single polypeptide chain comprising two monomers or core domains of one or two variant(s) according to the present invention or a combination of both.

Preferably the first and the second monomers are connected by a peptide linker.

It is understood that the scope of the present invention also encompasses the I-CreI variants, including heterodimers, obligate heterodimers, single chain meganuclease as non limiting examples, having at least one substitution in a position selected from the group 26, 28, 42.

According to another aspect of the present invention there is provided a polynucleotide fragment encoding the variant as defined above.

According to another aspect of the present invention there is provided an expression vector comprising at least one polynucleotide fragment as defined above.

The present invention also relates to a host cell, characterized in that it is modified by a polynucleotide or a vector according to the present invention.

The recombinant vectors comprising said polynucleotide may be obtained and introduced in a host cell by the well-known recombinant DNA and genetic engineering techniques.

The polypeptide of the invention may be obtained by culturing the host cell containing an expression vector comprising a polynucleotide sequence encoding said polypeptide, under conditions suitable for the expression of the polypeptide, and recovering the polypeptide from the host cell culture.

The present invention also relates to a non-human transgenic animal, characterized in that all or part of its constituent cells is modified by a polynucleotide or a vector according to the present invention.

The present invention also relates to a transgenic plant, characterized in that all or part of its constituent cells is modified by a polynucleotide or a vector according to the present invention.

The present invention also relates to the use of a meganuclease according to the present invention in a therapeutic method, in particular a meganuclease according to the present invention can be used for genome therapy ex vivo (gene cell therapy) and genome engineering. Most particularly the described meganucleases could be used to insert, delete or repair an endogenous or exogenous coding sequence.

To do this the meganuclease (or a polynucleotide encoding said meganuclease) and/or the targeting DNA are contained within a vector. Vectors comprising targeting DNA and/or nucleic acid encoding a meganuclease can be introduced into a cell by a variety of methods (e.g., injection, direct uptake, projectile bombardment, liposomes, electroporation). Meganucleases can be stably or transiently expressed into cells using expression vectors. Techniques of expression in eukaryotic cells are well known to those in the art. (See Current Protocols in Human Genetics: Chapter 12 “Vectors For Gene Therapy” & Chapter 13 “Delivery Systems for Gene Therapy”). Optionally, it may be preferable to incorporate a nuclear localization signal into the recombinant protein to be sure that it is expressed within the nucleus.

Once in a cell, the meganuclease and if present, the vector comprising targeting DNA and/or nucleic acid encoding the meganuclease are imported or translocated by the cell from the cytoplasm to the site of action in the nucleus. Whilst within the nucleus the meganuclease will cut any targets present in the genome and the vector resulting in double strand breaks which will be repaired by the endogenous repair mechanisms of the host cell and when a repair occurs between the genome and vector sequence this will result in a genome engineering event such as an insertion, deletion or repair.

For purposes of therapy, the meganucleases and a pharmaceutically acceptable excipient are administered in a therapeutically effective amount. Such a combination is said to be administered in a “therapeutically effective amount” if the amount administered is physiologically significant. An agent is physiologically significant if its presence results in a detectable change in the physiology of the recipient. In the present context, an agent is physiologically significant if its presence results in a decrease in the severity of one or more symptoms of the targeted disease and in a genome correction of the lesion or abnormality.

Definitions

Throughout the present Patent Application a number of terms and features are used to present and describe the present invention, to clarify the meaning of these terms a number of definitions are set out below and wherein a feature or term is not otherwise specifically defined or obvious from its context the following definitions apply.
Amino acid residues in a polypeptide sequence are designated herein according to the one-letter code, in which, for example, Q means Gln or Glutamine residue, R means Arg or Arginine residue and D means Asp or Aspartic acid residue.
Amino acid substitution means the replacement of one amino acid residue with another, for instance the replacement of an Arginine residue with a Glutamine residue in a peptide sequence is an amino acid substitution.
Altered/enhanced/increased cleavage activity, refers to an increase in the detected level of meganuclease cleavage activity, see below, against a target DNA sequence by a second meganuclease in comparison to the activity of a first meganuclease against the target DNA sequence. Normally the second meganuclease is a variant of the first and comprises one or more substituted amino acid residues in comparison to the first meganuclease.
by “beta-hairpin” it is intended two consecutive beta-strands of the antiparallel beta-sheet of a LAGLIDADG homing endonuclease core domain (β₁β₂ or β₃β₄) which are connected by a loop or a turn,
by “hybrid DNA target” or “non-palindromic DNA target” it is intended the fusion of a different half of two parent meganuclease target sequences. In addition at least one half of said target may comprise the combination of nucleotides which are bound by at least two separate subdomains (combined DNA target).
Cleavage activity: the cleavage activity of the variant according to the invention may be measured by any well-known, in vitro or in vivo cleavage assay, such as those described in the International PCT Application WO 2004/067736; Epinat et al., Nucleic Acids Res., 2003, 31, 2952-2962; Chames et al., Nucleic Acids Res., 2005, 33, e178; Arnould et al., J. Mol. Biol., 2006, 355, 443-458, and Arnould et al., J. Mol. Biol., 2007, 371, 49-65. For example, the cleavage activity of the variant of the invention may be measured by a direct repeat recombination assay, in yeast or mammalian cells, using a reporter vector. The reporter vector comprises two truncated, non-functional copies of a reporter gene (direct repeats) and the genomic (non-palindromic) DNA target sequence within the intervening sequence, cloned in a yeast or a mammalian expression vector. Usually, the genomic DNA target sequence comprises one different half of each (palindromic or pseudo-palindromic) parent homodimeric meganuclease target sequence. Expression of the heterodimeric variant results in a functional endonuclease which is able to cleave the genomic DNA target sequence. This cleavage induces homologous recombination between the direct repeats, resulting in a functional reporter gene (LacZ, for example), whose expression can be monitored by an appropriate assay. The specificity of the cleavage by the variant may be assessed by comparing the cleavage of the (non-palindromic) DNA target sequence with that of the two palindromic sequences cleaved by the parent homodimeric meganucleases or compared with wild type meganuclease.
by “selection or selecting” it is intended to mean the isolation of one or more meganuclease variants based upon an observed specified phenotype, for instance altered cleavage activity. This selection can be of the variant in a peptide form upon which the observation is made or alternatively the selection can be of a nucleotide coding for selected meganuclease variant.
by “screening” it is intended to mean the sequential or simultaneous selection of one or more meganuclease variant(s) which exhibits a specified phenotype such as altered cleavage activity.
by “derived from” it is intended to mean a meganuclease variant which is created from a parent meganuclease and hence the peptide sequence of the meganuclease variant is related to (primary sequence level) but derived from (mutations) the sequence peptide sequence of the parent meganuclease.
by “domain” or “core domain” it is intended the “LAGLIDADG homing endonuclease core domain” which is the characteristic α₁β₁β₂α₂β₃β₄α₃ fold of the homing endonucleases of the LAGLIDADG family, corresponding to a sequence of about one hundred amino acid residues. Said domain comprises four beta-strands (β₁β₂β₃β₄) folded in an antiparallel beta-sheet which interacts with one half of the DNA target. This domain is able to associate with another LAGLIDADG homing endonuclease core domain which interacts with the other half of the DNA target to form a functional endonuclease able to cleave said DNA target. For example, in the case of the dimeric homing endonuclease I-CreI (163 amino acids), the LAGLIDADG homing endonuclease core domain corresponds to the residues 6 to 94.
by “DNA target”, “DNA target sequence”, “target sequence”, “target-site”, “target”, “site”; “site of interest”; “recognition site”, “recognition sequence”, “homing recognition site”, “homing site”, “cleavage site” it is intended a 20 to 24 by double-stranded palindromic, partially palindromic (pseudo-palindromic) or non-palindromic polynucleotide sequence that is recognized and cleaved by a LAGLIDADG homing endonuclease such as I-CreI, or a variant, or a single-chain chimeric meganuclease derived from I-CreI. These terms refer to a distinct DNA location, preferably a genomic location, at which a double stranded break (cleavage) is to be induced by the meganuclease. The DNA target is defined by the 5′ to 3′ sequence of one strand of the double-stranded polynucleotide, as indicated for C1221 (see FIG. 1, SEQ ID NO: 41). Cleavage of the DNA target occurs at the nucleotides at positions +2 and −2, respectively for the sense and the antisense strand. Unless otherwise indicated, the position at which cleavage of the DNA target by an I-CreI meganuclease variant occurs, corresponds to the cleavage site on the sense strand of the DNA target.
by “DNA target half-site”, “half cleavage site” or half-site” it is intended the portion of the DNA target which is bound by each LAGLIDADG horning endonuclease core domain.
by “first/second/third/n^{th} series of variants” it is intended a collection of variant meganucleases, each of which comprises one or more amino acid substitution in comparison to a parent meganuclease from which all the variants in the series are derived.
by “functional variant” or “cutter” it is intended a variant which is able to cleave a DNA target sequence, preferably said target is a new target which is not cleaved by the parent meganuclease. For example, such variants have amino acid variation at positions contacting the DNA target sequence or interacting directly or indirectly with said DNA target.
by “heterodimer” it is intended to mean a meganuclease comprising two non-identical monomers. In particular the monomers may differ from each other in their peptide sequence and/or in the DNA target half-site which they recognise and cleave.
by “homologous” is intended a sequence with enough identity to another one to lead to a homologous recombination between sequences, more particularly having at least 95% identity, preferably 97% identity and more preferably 99%.
by “I-CreI” it is intended the wild-type I-CreI having the sequence of pdb accession code 1g9y, corresponding to the sequence SEQ ID NO: 51 in the sequence listing.
by “I-CreI variant with novel specificity” it is intended a variant having a pattern of cleaved targets different from that of the parent meganuclease. The terms “novel specificity”, “modified specificity”, “novel cleavage specificity”, “novel substrate specificity” which are equivalent and used indifferently, refer to the specificity of the variant towards the nucleotides of the DNA target sequence. In the present Patent Application the I-CreI variants described comprise an additional Alanine after the first Methionine of the wild type I-CreI sequence. These variants also comprise two additional Alanine residues and an Aspartic Acid residue after the final Proline of the wild type I-CreI sequence. These additional residues do not affect the properties of the enzyme and to avoid confusion these additional residues do not affect the numeration of the residues in I-CreI or a variant referred in the present Patent Application, as these references exclusively refer to residues of the wild type I-CreI enzyme as present in the variant, so for instance residue 2 of I-CreI is in fact residue 3 of a variant which comprises an additional Alanine after the first Methionine.
by “I-CreI site” it is intended a 22 to 24 by double-stranded DNA sequence which is cleaved by I-CreI. I-CreI sites include the wild-type (natural) non-palindromic I-CreI horning site (SEQ ID NO: 40) and the derived palindromic sequences such as the sequence 5′-t₋₁₂c₋₁₁a₋₁₀a₋₉a₋₈a₋₇c₋₆g₋₅t₋₄c₋₃g₋₂t₋₁a₊₁c₊₂g₊₃a₊₄c₊₅g₊₆t₊₇t₊₈t₊₉t₊₁₀g₊₁₁a₊₁₂ (SEQ ID NO: 41).
“identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default settings.
by “meganuclease”, it is intended an endonuclease having a double-stranded DNA target sequence of 12 to 45 bp. The nieganuclease is either a dimeric enzyme, wherein each domain is on a monomer or a monomeric enzyme comprising the two domains on a single polypeptide.
by “meganuclease domain”, it is intended the region which interacts with one half of the DNA target of a meganuclease and is able to associate with the other domain of the same meganuclease which interacts with the other half of the DNA target to form a functional meganuclease able to cleave said DNA target.
by “meganuclease variant” or “variant” it is intended a meganuclease obtained by replacement of at least one residue in the amino acid sequence of the parent meganuclease (natural or variant meganuclease) with a different amino acid.
by “monomer” it is intended to mean a peptide encoded by the open reading frame of the I-CreI gene or a variant thereof, which when allowed to dimerise forms a functional I-CreI enzyme. In particular the monomers dimerise via interactions mediated by the LAGLIDADG motif.
by “mutation” is intended the substitution, deletion, insertion of one or more nucleotides/amino acids in a polynucleotide (cDNA, gene) or a polypeptide sequence. Said mutation can affect the coding sequence of a gene or its regulatory sequence. It may also affect the structure of the genomic sequence or the structure/stability of the encoded mRNA.
Nucleotides are designated as follows: one-letter code is used for designating the base of a nucleoside: a is adenine, t is thymine, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a or t, v represents g, a or c, b represents g, t or c, h represents a, t or c, and n represents g, a, t or c.
by “peptide linker” it is intended to mean a peptide sequence of at least 10 and preferably at least 17 amino acids which links the C-terminal amino acid residue of the first monomer to the N-terminal residue of the second monomer and which allows the two variant monomers to adopt the correct conformation for activity and which does not alter the specificity of either of the monomers for their targets.
by “subdomain” it is intended the region of a LAGLIDADG homing endonuclease core domain which interacts with a distinct part of a homing endonuclease DNA target half-site.
by “single-chain meganuclease”, “single-chain chimeric meganuclease”, “single-chain meganuclease derivative”, “single-chain chimeric meganuclease derivative” or “single-chain derivative” it is intended a meganuclease comprising two LAGLIDADG homing endonuclease domains or core domains linked by a peptidic spacer. The single-chain meganuclease is able to cleave a chimeric DNA target sequence comprising one different half of each parent meganuclease target sequence.
by “targeting DNA construct/minimal repair matrix/repair matrix” it is intended to mean a DNA construct comprising a first and second portions which are homologous to regions 5′ and 3′ of the DNA target in situ. The DNA construct also comprises a third portion positioned between the first and second portion which comprise some homology with the corresponding DNA sequence in situ or alternatively comprise no homology with the regions 5′ and 3′ of the DNA target in situ. Following cleavage of the DNA target, a homologous recombination event is stimulated between the genome containing the NW genome and the repair matrix, wherein the genomic sequence containing the DNA target is replaced by the third portion of the repair matrix and a variable part of the first and second portions of the repair matrix.
by “vector” is intended a nucleic acid molecule capable of transporting another nucleic acid to which it has been linked, into a host cell in vitro, in vivo or ex vivo.

For a better understanding of the invention and to show how the same may be carried into effect, there will now be shown by way of example only, specific embodiments, methods and processes according to the present invention with reference to the accompanying drawings in which:

FIG. 1: Representation of some 22 bp DNA targets. The _P symbol stands for palindromic targets.

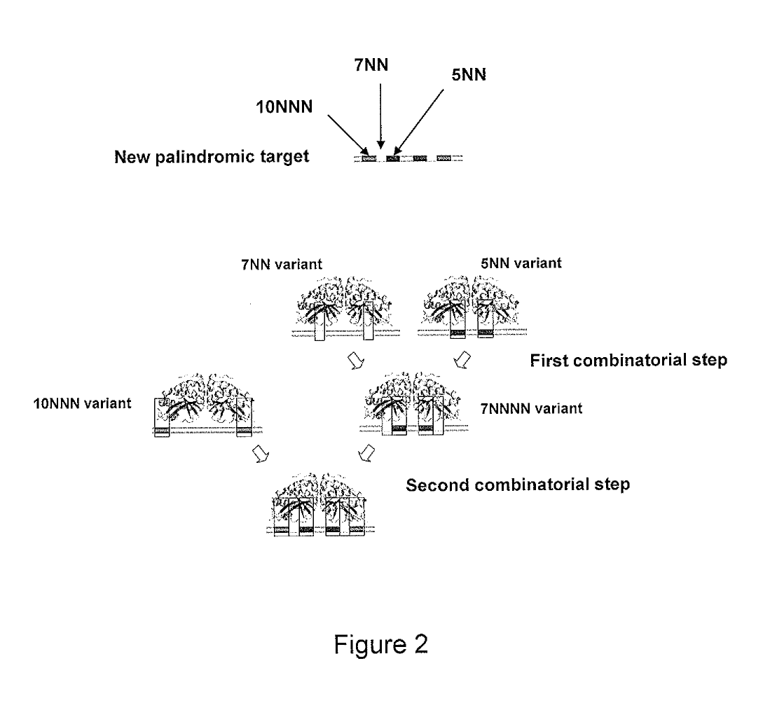

FIG. 2: Scheme of the engineering process of an I-CreI derived meganuclease directed to a DNA target where the nucleotides at positions ±10 to ±4 have been modified in comparison to C1221. The engineering process comprises two successive combinatorial steps.

FIG. 3: Structure of I-CreI in complex with its DNA target (PDB code 1G9Y). The structure is a zoom showing in particular the residues Gln26 and Lys28 that interact with the nucleotides at positions 7 and 6 of the C1234 target. Dashed lines represent hydrogen bonds

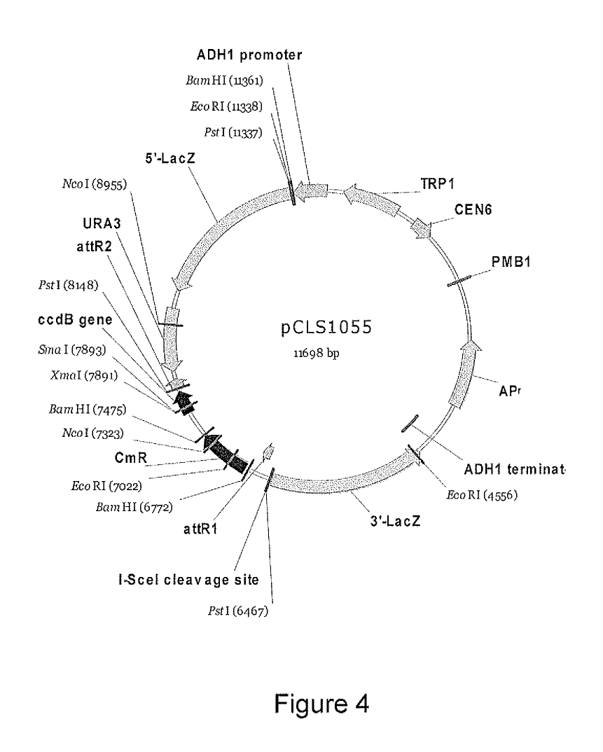

FIG. 4: pCLS1055 vector map

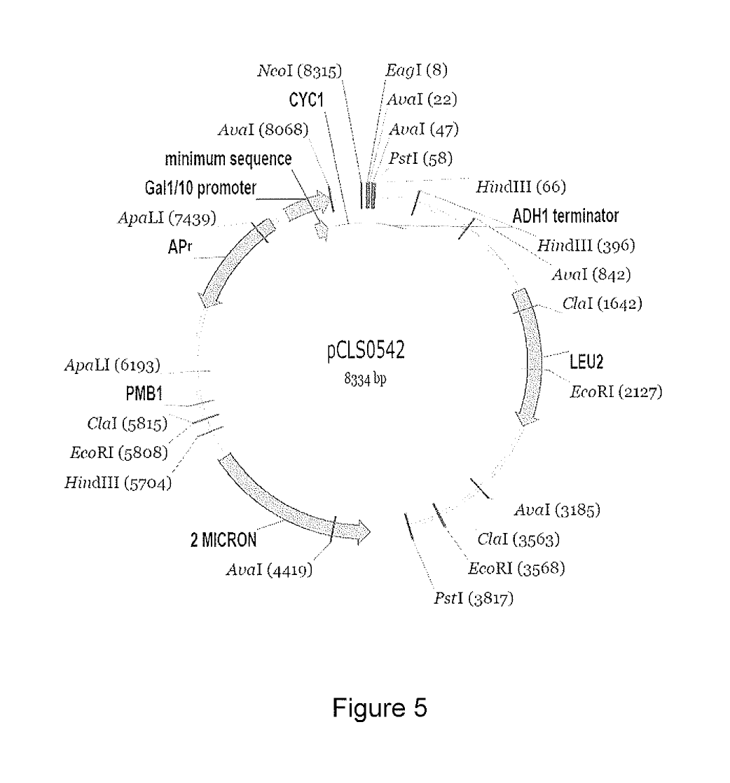

FIG. 5: pCLS0542 vector map

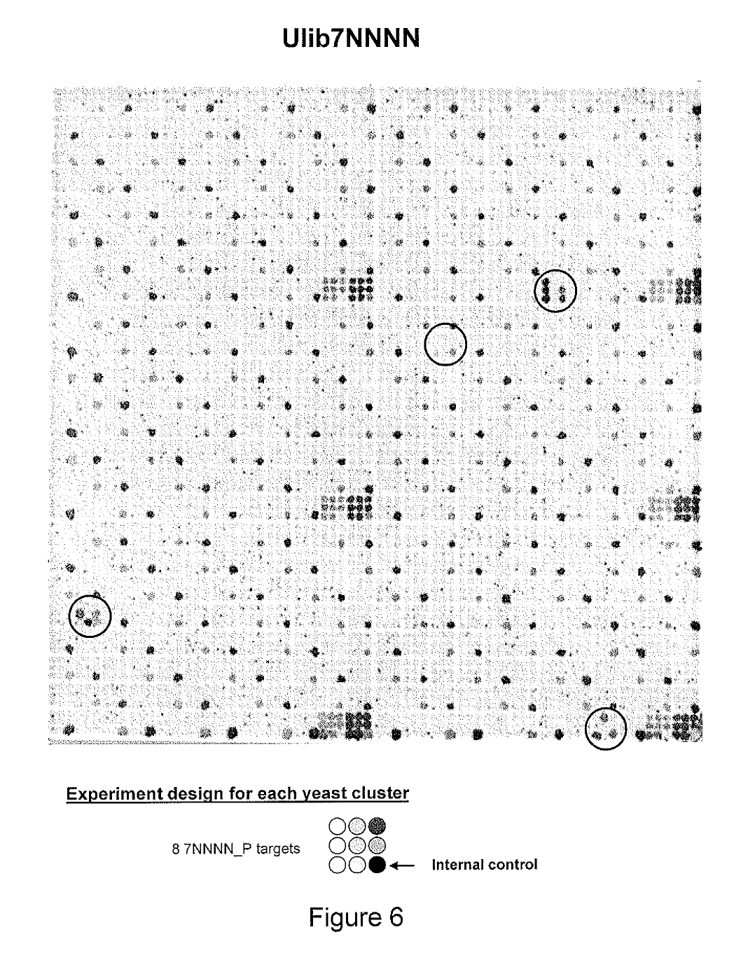

FIG. 6: Example of one yeast filter of the primary screening of the Ulib7NNNN variant library. The filter comprises six 96-well plates of Ulib7NNNN variants that have been screened against eight 7NNNN_P targets according to the experiment design. The four variants that show cleavage have been circled.

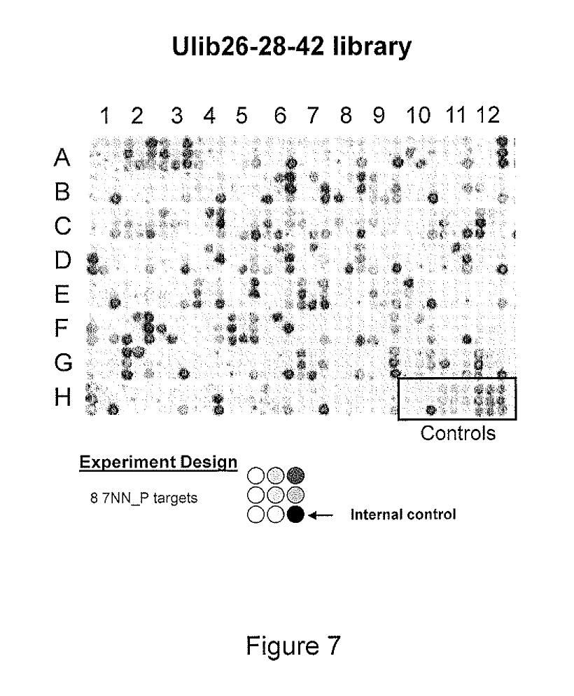

FIG. 7: The figure displays an example of secondary screening of Ulib26-28-42 variants against eight 7NN_P targets. Columns and rows are respectively noted from 1 to 12 and from A to H. In each 9-dots yeast cluster, an Ulib26-28-42 variant is screened against 8 different 7NN_P targets as exemplified by the experimental design. The bottom right dot is a cluster internal control. H10, H11 and H12 are also experiment controls.

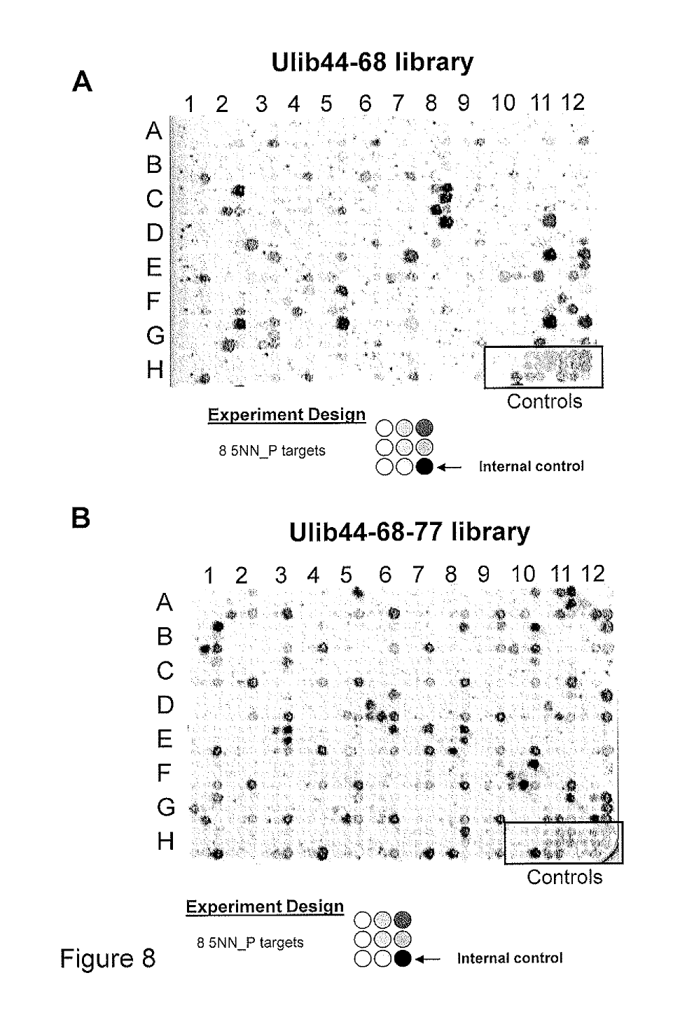

FIG. 8: A. The figure displays an example of primary screening of Ulib44-68 variants against eight 5NN_P targets. Columns and rows are respectively noted from 1 to 12 and from A to H. In each 9-dots yeast cluster, an Ulib44-68 variant is screened against 8 different 5NN_P targets as exemplified by the experimental design. The bottom right dot is a cluster internal control. H10, H11 and H12 are also experiment controls. B. The figure displays an example of primary screening of Ulib44-68-77 variants against eight 5NN_P targets. Columns and rows are respectively noted from 1 to 12 and from A to H. In each 9-dots yeast cluster, an Ulib44-68-77 variant is screened against 8 different 5NN_P targets as exemplified by the experimental design. The bottom right dot is a cluster internal control. H10, H11 and H12 are also experiment controls.

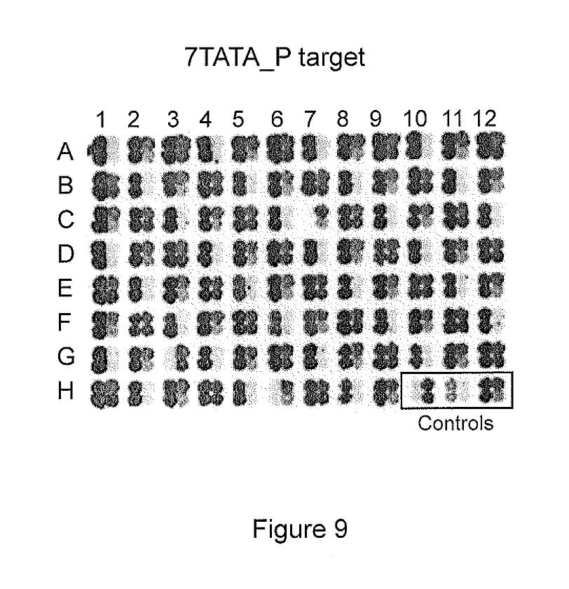

FIG. 9: The figure displays the secondary screening of the 96 rearranged combinatorial 7TATA_P I-CreI variants. Columns and rows are respectively noted from 1 to 12 and from A to H. In each 4-dots yeast cluster, the two left dots correspond to the same combinatorial variant, while the two right dots are experiment controls. H10, H11 and H12 are also experiment controls.

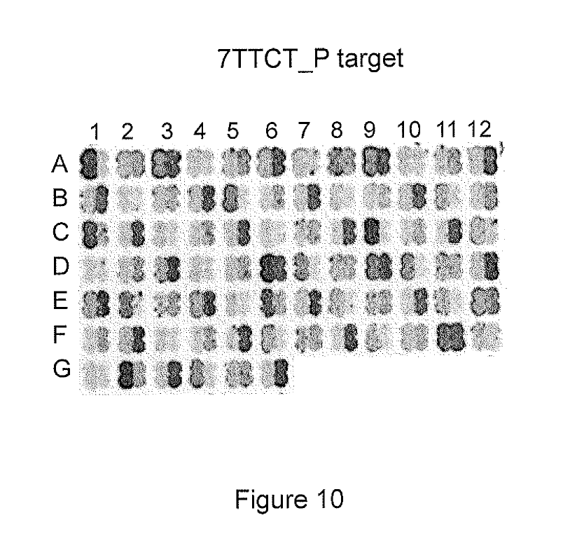

FIG. 10: The figure displays the secondary screening of the 78 positive combinatorial I-CreI variants that were found positive on the combined 7TTCT_P target. Columns and rows are respectively noted from 1 to 12 and from A to G. In each 4-dots yeast cluster, the two left dots correspond to the same combinatorial variant, while the two right dots are experiment controls.

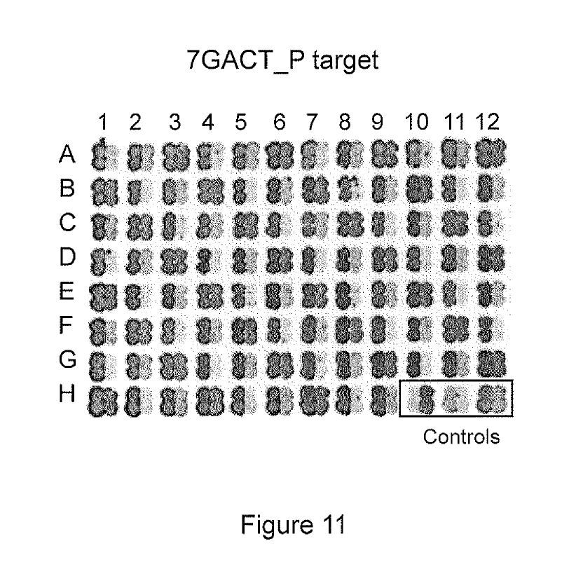

FIG. 11: The figure displays the secondary screening of the 96 rearranged combinatorial 7GACT_P I-CreI variants. Columns and rows are respectively noted from 1 to 12 and from A to H. In each 4-dots yeast cluster, the two left dots correspond to the same combinatorial variant, while the two right dots are experiment controls. H10, H11 and H12 are also experiment controls.

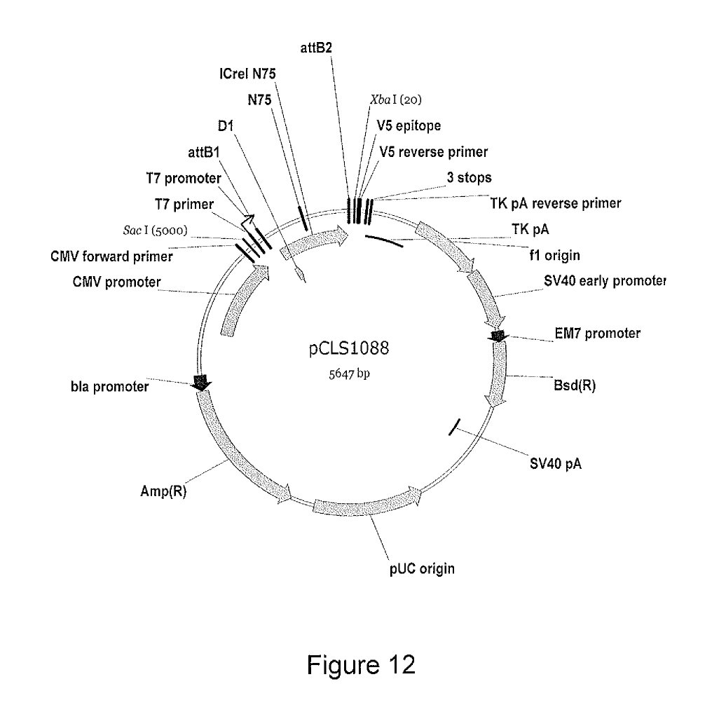

FIG. 12: pCLS1088 vector map.

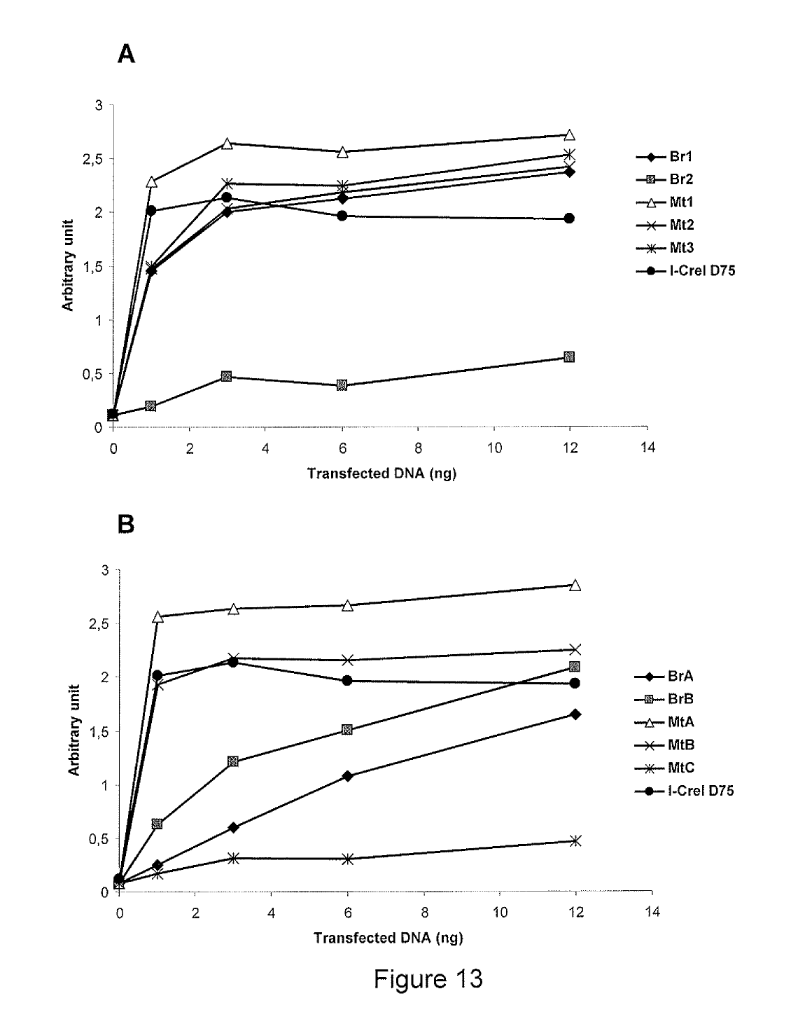

FIG. 13: Extrachromosomal SSA assay in CHO-K1 cells. A. The Br1, Br2, and Mt1 to Mt3 variants have been probed for cleavage of the 7TATA_P target in a dose—response manner. The activity cleavage of I-CreI against C1221 is shown as a positive control. B. The BrA, BrA, and MtA to MtC variants have been probed for cleavage of the 7GACT_P target in a dose—response manner. The activity cleavage of I-CreI against C1221 is shown as a positive control.

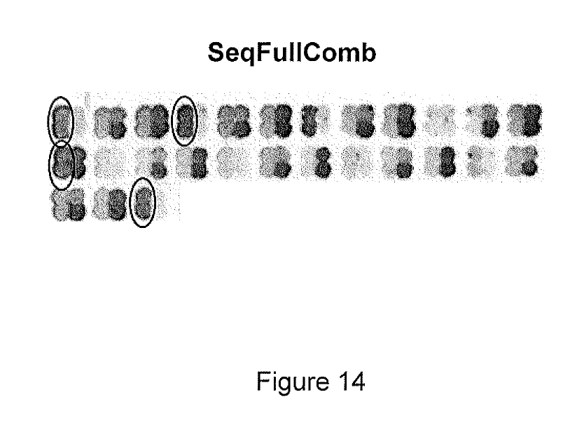

FIG. 14: Secondary screening of the 27 clones of the SeqFullComb library that had been selected for the FullComb_P target cleavage. In each 4-dots yeast cluster, the two left dots correspond to the same SegFullComb variant, while the two right dots are experiment controls. The four variants called FC1 to FC4 (Table 6) have been circled.

There will now be described by way of example a specific mode contemplated by the Inventors. In the following description numerous specific details are set forth in order to provide a thorough understanding. It will be apparent however, to one skilled in the art, that the present invention may be practiced without limitation to these specific details. In other instances, well known methods and structures have not been described so as not to unnecessarily obscure the description.

### EXAMPLE 1

### Engineering of Meganucleases Derived from I-CreI with an Altered Specificity Toward the 7NN Region

In this example, the inventors successfully altered the 7NN specificity of the I-CreI protein. For that purpose, a variant library was built in yeast where I-CreI residues Gln26, Lys28 and Thr42 were randomized. Analysis of the structure of I-CreI in complex with its DNA target shows that residues Lys28 and Gln26 interact respectively with the bases at positions 7 and 6 of the target complementary strand. In addition, the residue Thr42 located on the β3 β-strand of I-CreI is oriented toward the 7NN region (FIG. 3). The mutation of Thr42 by an amino acid with a longer side chain could hence promote an interaction with the 7NN region. This variant library was then screened against the sixteen 7NN_P targets.

### Material and Methods

### a) Construction of the Sixteen 7NN_P Target Vectors

The 7NN _P targets (FIG. 1) were cloned as follows: an oligonucleotide corresponding to the target sequence flanked by gateway cloning sequence was ordered from Proligo (5′-TGGCATACAAGTTTTCAAANNGTCGTACGACNNTTTGACAATCGTCTG TCA-3′, SEQ ID NO: 1). Double-stranded target DNA, generated by PCR amplification of the single stranded oligonucleotide, was cloned using the Gateway protocol (Invitrogen) into yeast reporter vector (pCLS1055). Yeast reporter vector was transformed into S. cerevisiae strain FYBL2-7B (MAT α, ura3 [symbol] 851, trp1 [symbol] 63, leu2 [symbol] 1, lys2 [symbol] 202).

### b) Generation of the Ulib26-28-42 Variant Library

In order to generate I-CreI derived coding sequences with the randomization of residues at positions 26, 28 and 42, two separate overlapping PCR reactions were carried out that amplify the 5′ end (aa positions 1-37) or the 3′ end (positions 32-167) of the I-CreI coding sequence. For the 5′ end, PCR amplification is carried out using the Gal10F primer (SEQ ID NO: 2) and the Ulib7NNRev (SEQ ID NO: 3). For the 3′ end, PCR amplification is carried out using the Gal10R primer (SEQ ID NO: 4) and a primer specific to the I-CreI coding sequence for amino acids 32-46 (Ulib7NNFor: 5′ tcttataagtttaaacatcagctaagcttgnvktttcaggtgact-3′, SEQ ID NO: 5). Then, to generate the variant library called Ulib26-28-42, 25 ng of each of the two overlapping PCR fragments and 75 ng of vector DNA (pCLS0542) linearized by digestion with NcoI and EagI were used to transform the yeast Saccharomyces cerevisiae strain FYC2-6A (MATα, trp1Δ63, leu2Δ1, his3Δ200) using a high efficiency LiAc transformation protocol (Gietz and Woods 2002). An intact coding sequence containing the mutations is generated by in vivo homologous recombination in yeast. 2232 clones were picked for further experiment. They represent 66% of the theoretical protein diversity of Ulib26-28-42.

### Results

The 2232 clones from the Ulib26-28-42 variant library were screened for cleavage against the sixteen 7NN_P targets using our yeast screening assay. The primary screening yielded 836 positive clones that cleave at least one target. All the sixteen targets were cleaved at least by one variant. 465 positive clones were rearranged, sequenced and processed again for a secondary screening (FIG. 7). The sequencing resulted in 266 unique variant sequences and the 16 7NN_P targets are cleaved by at least one variant from the Ulib26-28-42 library.

### EXAMPLE 2

### Engineering of Meganucleases Derived from I-CreI with an Altered Specificity Toward 5NN Nucleotides

To change the specificity of I-CreI toward the 5NN nucleotides, two variant libraries were generated in yeast: Ulib44-68 by randomizing residues Gln44, Arg68 and Ulib44-68-77 by randomizing residues Gln44, Arg68 and Ile77. Residue Gln44 interacts with the base at position 4 of the target complementary strand, Arg68 interacts with the nucleotide at position 5 and Ile77 is oriented toward nucleotides at positions 6 and 5 of the DNA target. Both libraries were screened against the sixteen 5NN_P targets.

### Material and Methods

### a) Construction of the Sixteen 5NN_P Target Vectors

The 5NN_P targets were cloned as follows: an oligonucleotide corresponding to the target sequence flanked by gateway cloning sequence was ordered from Proligo (5′-TGGCATACAAGTTTTCAAAACNNCGTACGNNGTTTTGACAATCGTCTG TCA-3′, SEQ ID NO: 6). Double-stranded target DNA, generated by PCR amplification of the single stranded oligonucleotide, was cloned using the Gateway protocol (Invitrogen) into yeast reporter vector (pCLS1055). Yeast reporter vector was transformed into S. cerevisiae strain FYBL2-7B (MAT α, ura3 [symbol] 851, trp1 [symbol] 63, leu2 [symbol] 1, lys2 [symbol] 202).

### b) Generation of the Ulib44-68 Variant Library

In order to generate I-CreI derived coding sequences with the randomization of residues at positions 44 and 68, two separate overlapping PCR reactions were carried out that amplify respectively the residues 1 to 59 and the residues 54 to 167 of the I-CreI coding sequence. The first PCR fragment was amplified using the primers Gal10F (SEQ ID NO: 2) and Cre44Rev (5′-cactagtttgtccagaaaccaacggcgctgggtatttgagtcacmnriaaaggtcaagct-3′, SEQ ID NO: 7), and the second fragment with the Cre68For primer (5′-tactggacaaaetagtggatgaaattggegttggttaegtannkgatcgeggatec-3′, SEQ ID NO: 8) and Gal10R (SEQ ID NO: 4) primers. To generate the variant library called Ulib44-68, 25 ng of each PCR fragment and 75 ng of vector DNA (pCLS0542) linearized by digestion with NcoI and EagI were used to transform the yeast Saccharomyces cerevisiae strain FYC2-6A (MATα, trp1Δ63, leu2Δ1, his3Δ200) using a high efficiency LiAc transformation protocol (Gietz and Woods 2002). An intact coding sequence containing the mutations is generated by in vivo homologous recombination in yeast. 1116 clones were picked for further experiment, representing 2.8 times the theoretical protein diversity of Ulib44-68.

### c) Generation of the Ulib44-68-77 Variant Library

In order to generate I-CreI derived coding sequences with the randomization of residues at positions 44, 68 and 77, three separate overlapping PCR reactions were carried out that amplify respectively the residues 1 to 43, the residues 37 to 67 and the residues 63 to 167 of the I-CreI coding sequence. The first PCR fragment was amplified using the primers Gal10F (SEQ ID NO: 2) and Cre43Rev (5′-aaaggtcaagettagctgatgataaa-3′, SEQ ID NO: 9), the second fragment with the Cre44For (5′-catcagctaagcttgacctttnnkgtgactcaaaagacc-3′, SEQ ID NO: 10) and Cre67Rev (SEQ ID NO: 11) primers, and the third fragment with the Cre68-77For (SEQ ID NO: 12) and Gal10R (SEQ ID NO: 4) primers. Before transforming the yeast strain, an assembly PCR was performed with the two first PCR fragments using the Gal10R (SEQ ID NO: 4) and Cre67Rev (SEQ ID NO: 11) primers. Then, to generate the variant library called Ulib44-68-77, 25 ng of each of the assembly PCR fragment and the Cre68-77For-Gal10R PCR fragment and 75 ng of vector DNA (pCLS0542) linearized by digestion with NcoI and EagI were used to transform the yeast Saccharomyces cerevisiae strain FYC2-6A (MATα, trp1Δ63, leu2Δ1, his3Δ200) using a high efficiency LiAc transformation protocol (Gietz and Woods 2002). An intact coding sequence containing the mutations is generated by in vivo homologous recombination in yeast. 2232 clones were picked for further experiment. They represent 28% of the theoretical protein diversity of Ulib44-68-77.

### Results

The 1116 clones constituting the Ulib44-68 library were screened against the sixteen 5NN_P targets using our yeast screening assay (FIG. 8A). The primary screening yielded 458 positive clones that cleave at least one 5NN_P target, thirteen 5NN_P targets being cleaved at least once. No cutters were obtained for the three 5AG_P, 5CG_P and 5TG_P targets. The positive clones were rearranged and sequenced to obtain 189 unique variant sequences.

The 2232 clones constituting the Ulib44-68-77 library were screened against the sixteen 5NN_P targets using the yeast screening assay described above (FIG. 8B). The primary screening yielded 980 positive clones that cleave at least one 5NN_P target, all the sixteen targets being cleaved at least once. All the positive clones were rearranged and sequenced. The sequencing resulted in 493 unique variant sequences.

### EXAMPLE 3

### Making of Meganucleases Cleaving the 7TATA_P Target Using a Combinatorial Method

The 7TATA_P target is a combination of the 5TA_P and 7TA_P targets (FIG. 1). Variants able to cleave the 7TA_P or the 5TA_P targets have been obtained as described in the previous examples 1 and 2. They belong respectively to the Ulib26-28-42 and Ulib44-68 or Ulib44-68-77 variant libraries. In this example, the inventors show how to combine mutations at positions 44, 68 and 77 from proteins cleaving the 5TA_P target (CAAAACTACGT_P) with mutations at positions 26, 28 and 42 from proteins cleaving the 7TA_P target (CAAATAGTCGTP) to check whether combined variants could cleave the 7TATA_P target (CAAATATACGT_P).

### Material and Methods

### a) Construction of Combinatorial Variants

I-CreI variants cleaving the 7TA_P or 5TA_P targets were identified previously. In order to generate I-CreI derived coding sequence containing mutations from both series, separate overlapping PCR reactions were carried out that amplify the 5′ end (aa positions 1-50) of variants from the Ulib26-28-42 library or the 3′ end (positions 43-167) of variants belonging to the Ulib44-68-77 library. For both the 5′ and 3′ end, PCR amplification is carried out using the Gal10F (SEQ ID NO: 2) and Gal10R (SEQ ID NO: 4) primers specific to the vector and primers specific to the I-CreI coding sequence for amino acids 43-50: Comb75assFor (5′-tttXXXgtgactcaaaagacccag-3′, SEQ ID NO: 13) and Comb75assRev (5′-ctgggtettttgagtcacXXXaaa-3′, SEQ ID NO: 14) where XXX codes for residue 44. The PCR fragments resulting from the amplification reaction realized with the same primers and with the same coding sequence for residue 44 were pooled. Then, each pool of PCR fragments resulting from the reaction with primers Gall OF (SEQ ID NO: 2) and Comb75assRev (SEQ ID NO: 14, for Ulib26-28-42 variants) or Comb75assFor (SEQ ID NO: 13) and Gal10R (SEQ ID NO: 4, for Ulib44-68-77 variants) was mixed in an equimolar ratio. Finally, approximately 25 ng of each final pool of the two overlapping PCR fragments and 75 ng of vector DNA (pCLS0542) linearized by digestion with NcoI and EagI were used to transform the yeast Saccharomyces cerevisiae strain FYC2-6A (MATα, trp1Δ63, leu2Δ1, his3Δ200) using a high efficiency LiAc transformation protocol (Gietz and Woods 2002). An intact coding sequence containing both groups of mutations is generated by in vivo homologous recombination in yeast.

### Results

I-CreI combinatorial variants were constructed by associating mutations at positions 44, 68 and 77 of twenty 5TA_P cutters coming from Ulib44-68 or Ulib44-68-77 with mutations at positions 26, 28 and 42 of twenty 7TA_P cutters coming from Ulib26-28-42. The resulting combinatorial library has a complexity of 400 variants. This library was transformed into yeast and 1116 clones (2.8 times the diversity) were screened for cleavage against the 7TATA_P DNA target. 714 clones of the combinatorial 7TATA library turned out to be positive. Only 93 clones were rearranged and sequenced. They yielded 55 unique sequences corresponding to novel combinatorial meganucleases. An example of such meganucleases is given in Table 1. The secondary screening confirmed their strong cleavage efficacy against the 7TATA_P target (FIG. 9).

TABLE 1
Panel of variants theoretically presents in the 7TATA combinatorial library
used in example 3. (Only 72 out of the 400 possible combinations are displayed).

| | | Amino acids at positions 44, 68 and77 | | | | | | | | |
|---|---|---|---|---|---|---|---|---|---|---|
| | | (ex: VMR stands for 44V68M77R) | | | | | | | | |
| | | VRI | VMR | VKI | VHV | CRI | CYL | CQH | ILH | IRI |
| Amino acids at positions | WTR | + | | | + | + | | | | + |
| 26, 28 and 42 | TTR | + | | | | + | | | | |
| (ex: WTR stands for | CRT | + | | | | | | | | |
| 26W28T42R) | CSR | | | | | + | | | | |
| | WAR | + | | + | | + | | | | + |
| | CAK | | | | | | | | | |
| | STR | + | | | | + | + | | | + |
| | ASR | | | | | | | | | + |

+ indicates that the combinatorial variant was found among the sequenced positives.

### EXAMPLE 4

### Making of Meganucleases Cleaving the 7TTCT_P Target Using a Combinatorial Method

The 7TTCT_P target is a combination of the 5CT_P and 7TT_P targets (FIG. 1). Variants able to cleave the 7TT_P or the 5CT_P targets have been obtained as described in the previous examples 1 and 2. They belong respectively to the Ulib26-28-42 and Ulib44-68 or Ulib44-68-77 variant libraries. In this example, the inventors show how to combine mutations at positions 44, 68 and 77 from proteins cleaving the 5CT_P target (CAAAACCTCGT_P) with mutations at positions 26, 28 and 42 from proteins cleaving the 7TT_P target (CAAATTGTCGT_P) to check whether combined variants could cleave the 7TTCT_P target (CAAATTCTCGT_P).

### Material and Methods

As described in example 3.

### Results

I-CreI combinatorial variants were constructed by associating mutations at positions 44, 68 and 77 of 34 5CT_P cutters coming from Ulib44-68 or Ulib44-68-77 with mutations at positions 26, 28 and 42 of 16 7TT_P cutters coming from Ulib26-28-42. The resulting combinatorial library has a complexity of 544 variants. This library was transformed into yeast and 1116 clones (2 times the diversity) were screened for cleavage against the 7TTCT_P DNA target. The primary screening allowed for obtaining 78 positive clones, which were rearranged and sequenced. These 78 positive clones correspond to 34 unique sequences of novel combinatorial meganucleases. A panel of such meganucleases derived from I-CreI is given in Table 2. The cleavage of the 7TTCT_P target was confirmed by a secondary screening (FIG. 10).

TABLE 2
Panel of variants theoretically presents in the 7TTCT combinatorial library
used in example 4. (Only 72 out of the 544 possible combinations are displayed).

| | | Amino acids at positions 44, 68 and77 | | | | | | | | |
|---|---|---|---|---|---|---|---|---|---|---|
| | | (ex: KYN stands for 44K68Y77N) | | | | | | | | |
| | | QTL | QYL | QCI | KSI | RTI | RSI | KYN | KTI | KAQ |
| Amino acids at positions | TTR | | | | | | | | | |
| 26, 28 and 42 | CSR | | + | | + | | | | | |
| (ex: TTR stands for | WAR | | | | | | | | | |
| 26T28T42R) | CAR | | | | | + | | | | |
| | SAR | | | | | | | | | |
| | STR | | + | | + | | | | + | |
| | HRT | | | | | | | | | |
| | ARS | | + | | + | | | | | + |

+ indicates that the combinatorial variant was found among the sequenced positives.

### EXAMPLE 5

### Making of Meganucleases Cleaving the 7GACT_P Target Using a Combinatorial Method

The 7GACT P target is a combination of the 5CT_P and 7GA_P targets (FIG. 1). Variants able to cleave the 7GA_P or the 5CT_P targets have been obtained as described in the previous examples 1 and 2. They belong respectively to the Ulib26-28-42 and Ulib44-68 or Ulib44-68-77 variant libraries. In this example, the inventors show how to combine mutations at positions 44, 68 and 77 from proteins cleaving the 5CT_P target (CAAAACCTCGT_P) with mutations at positions 26, 28 and 42 from proteins cleaving the 7GA_P target (CAAAGAGTCGT_P) to check whether combined variants could cleave the 7GACT_P target (CAAAGACTCGT_P).

### Material and Methods

As described in example 3.

### Results

I-CreI combinatorial variants were constructed by associating mutations at positions 44, 68 and 77 of 34 variants coming from Ulib44-68 or Ulib44-68-77 that cleave the 5CT_P target (the same variants that were used for example 5) with mutations at positions 26, 28 and 42 of 15 variants coming from Ulib26-28-42 that cleave the 7GA_P target. The resulting combinatorial library has a complexity of 510 variants. This library was transformed into yeast and 1116 clones (2.2 times the diversity) were screened for cleavage against the 7GACT_P DNA target. The primary screening allowed for obtaining 850 positive clones. The 93 positive clones that gave the strongest signal for cleavage were rearranged and sequenced, yielding to 60 confirmed unique variant sequences of novel combinatorial meganucleases. A panel of such meganucleases derived from I-CreI is given in Table 3. The strong cleavage of the 7GACT_P target was confirmed by a secondary screening (FIG. 11).

TABLE 3
Panel of variants theoretically presents in the 7GACT combinatorial library
used in example 5. (Only 72 out of the 510 possible combinations are displayed).

| | | Amino acids at positions 44, 68 and77 | | | | | | | | |
|---|---|---|---|---|---|---|---|---|---|---|
| | | (ex: RCT stands for 44R68C77T) | | | | | | | | |
| | | RAI | KSI | KVI | RCT | QVH | KAQ | RCI | QYL | KYN |
| Amino acids at positions | ANR | + | | | | | | | | |
| 26, 28 and 42 | CAK | | + | | | | | | | |
| (ex: SAR stands for | CNK | | | | | | | | | |
| 26S28A42R) | SAR | | | + | | | | | | |
| | SNR | | | + | | | | | | |
| | STR | + | + | + | + | + | | | | |
| | TTK | | | | | | | | | + |
| | TTR | | + | | | | + | + | | + |

+ indicates that the combinatorial variant was found among the sequenced positives.

### EXAMPLE 6

### Making of Engineered Meganucleases Derived From I-CreI that Cleave 7NNNN_P Targets by Screening a High Diversity Variant Library

In this example, the inventors show how they were able to generate directly 7NNNN_P cutters by screening a high diversity variant library in yeast. This library was built by randomizing residues at positions 26, 28, 42, 44, 68 and 77 and screened against the 256 7NNNN_P targets.

### Material and Methods

### a) Construction of the 256 7NNNN_P Target Vectors

The 7NNNN_P targets (FIG. 1) were cloned as follows: an oligonucleotide corresponding to the target sequence flanked by gateway cloning sequence was ordered from Proligo (5′-TGGCATACAAGTTTTCAAANNNNCGTACGACNNNNTGACAATCGTCTG TCA-3′, SEQ ID NO: 15). Double-stranded target DNA, generated by PCR amplification of the single stranded oligonucleotide, was cloned using the Gateway protocol (Invitrogen) into yeast reporter vector (pCLS1055, FIG. 4). Yeast reporter vector was transformed into S. cerevisiae strain FYBL2-7B (MAT α, ura3 [symbol] 851, trp1 [symbol] 63, leu2 [symbol] 1, lys2 [symbol] 202).

b) Generation of the Ulib7NNNN variant library

In order to generate I-CreI derived coding sequences with the randomization of residues at positions 26, 28, 42, 44, 68 and 77, three separate overlapping PCR reactions were carried out that amplify respectively the residues 1 to 37, the residues 32 to 67 and the residues 63 to 167 of the I-CreI coding sequence. The first PCR fragment was amplified using the primers Gal10F (5′-GCAACTTTAGTGCTGACACATACAGG-3′, SEQ ID NO: 2) and Ulib7NNRev (5′-atgataaacttataagactggtttggmbnaatmbnagegatgatgct-3′, SEQ ID NO: 3), the second fragment with the Ulib7NNForBis (5′-tatataagtttaaacatcagetaagettgnvkittnnkgtgactcaaaag-3′, SEQ ID NO: 5) and Cre67Rev (5′-tacgtaaccaacgccaatttcatccac-3′, SEQ ID NO: 11) primers, and the third fragment with the Cre68-77For (5′-ggcgttggttacgtannkgatcgcggatccgatcegattaennkttaagegaaatc-3′, SEQ ID NO: 12) and Gal10R (5′-ACAACCTTGATTGGAGACTTGACC-3′, SEQ ID NO: 4) primers.

The nvk code in the oligonucleotides allows the degeneracy at the positions 26, 28 and 42 among the 15 following amino acids: A, C, D, E, G, H, K, N P, Q, R, 5, T, W Y. The nnk code in the oligonucleotides allows the degeneracy at the positions 44, 68 and 77 among the 20 possible amino acids Before transforming the yeast strain, an assembly PCR was performed with the two first PCR fragments using the Gal10R and Cre67Rev primers. Then, to generate the Ulib7NNNN variant library, 25 ng of each of the assembly PCR fragment and the Cre68-77For PCR fragment and 75 ng of vector DNA pCLS0542 (FIG. 5) linearized by digestion with NcoI and EagI were used to transform the yeast Saccharomyces cerevisiae strain FYC2-6A (MATα, trp1 Δ63, leu2Δ1, his3Δ200) using a high efficiency LiAc transformation protocol (Gietz and Woods 2002). An intact coding sequence containing the mutations is generated by in vivo homologous recombination in yeast. 4464 clones were picked for further experiment. They represent 0.02% of the theoretical protein diversity of Ulib7NNNN.

### c) Mating of Meganuclease Expressing Clones and Screening in Yeast

Screening was performed as described previously (Arnould, Chames et al. 2006). Mating was performed using a colony gridder (QpixII, Genetix). Variants were gridded on nylon filters covering YPD plates, using a low gridding density (about 4 spots/cm²). A second gridding process was performed on the same filters to spot a second layer consisting of different reporter-harboring yeast strains for each target. Membranes were placed on solid agar YPD rich medium, and incubated at 30° C. for one night, to allow mating. Next, filters were transferred to synthetic medium, lacking leucine and tryptophan, with galactose (1%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH 7.0, 0.1% SDS, 6% dimethyl formamide (DMF), 7 mM β-mercaptoethanol, 1% agarose, and incubated at 37° C., to monitor β-galactosidase activity. Results were analyzed by scanning and quantification was performed using proprietary software.

### Results

The 4464 clones constituting the Ulib7NNNN library were screened against the 256 (4⁴) 7NNNN_P targets using our yeast screening assay (FIG. 6). The primary screening yielded 436 positive clones that cleave at least one 7NNNN_P target. Overall, the screening showed the cleavage of 159 7NNNN_P targets among the 256 targets. All the positive clones were rearranged and sequenced. The sequencing resulted in 305 unique variant sequences.

### EXAMPLE 7

### Cleavage Activity Comparison Between Variants from the Ulib7NNNN Library And Combined 7NN×5NN Variants

Some of the variants that were isolated during the primary screening of the Ulib7NNNN library (Example 6) had saturating activities in yeast toward the 7TATA_P or 7GACT_P targets like some of the variants that were obtained in examples 3 or 5. To compare the cleavage activity of different variants that were obtained by the two processes (either by the screening of the Ulib7NNNN library or by the 7NN×5NN combinatorial process), they were further evaluated using an extrachromosomal SSA assay in CHO-K1 cells.

### Material and Methods

a) Recloning of I-CreI Derived Variants into a Mammalian Expression Vector

The variant ORF was amplified by PCR using the primers CCM2For (5′-aagcagagetetctggetaactagagaacecactgettaetggettategaccatggccaataccaaatataacaaag agttec-3′: SEQ ID NO: 17) and CCMRevBis (5′-CTGCTCTAGATTAGTCGGCCGCCGGGGAGGATTICTTC-3′: SEQ ID NO: 18). The PCR fragment was digested by the restriction enzymes SacI and XbaI, and was then ligated into the vector pCLS1088 (FIG. 12) digested also by SacI and XbaI. Meganuclease expression is driven by a CMV promoter.

### b) Extrachromosomal SSA Assay in Mammalian Cells

CHO-K1 cells were transfected with 200 ng of DNA containing various amounts of meganuclease expression vectors (0 to 12 ng) and 150 ng of the reporter plasmid, in the presence of Polyfect transfection reagent in accordance with the manufacturer's protocol (Qiagen). The culture medium was removed 72 hours after transfection, and 150 μl of lysis/detection buffer was added for β-galactosidase liquid assay (typically, for 1 liter of buffer, we used 100 ml of lysis buffer (10 mM Tris-HCl pH7.5, 150 mM NaCl, 0.1% Triton X100, 0.1 mg/ml BSA, protease inhibitors), 10 ml of Mg 100× buffer (MgCl₂ 100 mM, 2-mercaptoethanol 35%), 110 ml of an 8 mg/ml solution of ONPG and 780 ml of 0.1M sodium phosphate pH7.5). After incubation at 37° C., we measured optical density at 420 nm. The entire process was performed on 96-well plate format using an automated Velocity11 BioCel platform

### Results

Table 4 indicates the variants that were subcloned into a mammalian expression vector and further submitted to an extrachromosomal SSA aasy in CHO-K1 cells.

TABLE 4
Variants that were further characterized by an extrachromosomal
SSA assay in CHO-K1 cells.

| | | | | SEQ |
|---|---|---|---|---|
| | | Cleaved | | ID |
| Variants | Variant Origin | target | Sequence | NO: |
| Br1 | Ulib7NNNN | 7TATA_P | 26A28T42K44V77H | 19 |
| Br2 | Ulib7NNNN | 7TATA_P | 26Y28S42R44V68L77L | 20 |
| Mt1 | Combinatorial | 7TATA_P | 26W28A42R44I68Y | 21 |
| | 7TATA library | | | |
| Mt2 | Combinatorial | 7TATA_P | 26W28T42R44I68Y77L | 22 |
| | 7TATA library | | | |
| Mt3 | Combinatorial | 7TATA_P | 26C28S42R44V68Y77L | 23 |
| | 7TATA library | | | |
| BrA | Ulib7NNNN | 7GACT_P | 26T28T42S44R77K | 24 |
| BrB | Ulib7NNNN | 7GACT_P | 26C28S42R44R68S77C | 25 |
| MtA | Combinatorial | 7GACT_P | 26S28T42R44R68Y77N | 26 |
| | 7GACT library | | | |
| MtB | Combinatorial | 7GACT_P | 26T28T42R44K54L68Q | 27 |
| | 7GACT library | | | |
| MtC | Combinatorial | 7GACT_P | 26T28T42R44K68A77Q | 28 |
| | 7GACT library | | | |

FIG. 13 shows the cleavage efficiency of the variants described in table 4 against their respective target. In each experiment, the cleavage profile of C1221 by I-CreI D75 (the wild-type I-CreI protein) is shown as a positive control. The Br1 variant that has been isolated through the Ulib7NNNN screening matches in terms of cleavage activity three 7TATA_P cutters that have been obtained through a combinatorial process described in example 4 as well as the wild-type I-CreI (FIG. 13A). FIG. 13B shows that activity of MtA even exceeds that of I-CreI. Activity of BrA and BrB that have been obtained with the Ulib7NNNN screening is similar to the activity of I-CreI and MtB at 12 ng of transfected expression vector.

These results demonstrate that I-CreI derived variants able to cleave 7NNNN_P targets can be generated directly with the screening of a variant library and that some of these cutters can be compared in terms of cleavage activity to variants that have been obtained through a combinatorial process as described in examples 3 to 5.

### EXAMPLE 8

### Making of Engineered I-CreI Derived Meganucleases with an Altered Specificity Toward Nucleotides ±10 to ±4

In the present example the inventors engineer an I-CreI variant with a modified specificity toward nucleotides ±10 to ±4 as shown in FIG. 2. The FullComb_P palindromic DNA sequence (FIG. 1) is a combination of the 10TTG_P and 7GACT_P targets. To engineer I-CreI variants able to cleave the FullComb_P target, a sequential combinatorial approach was chosen (WO2010015899). Nevertheless, the combinatorial method described in WO2007/049095 can also be used. Ulib7NNNN variants able to cleave the 7GACT_P target as described in example 5 were chosen to build a sequential variant library where residues at positions 32, 33 and 38 were randomized.

Material and methods

### a) Construction of the Sequential Variant Library SeqFullComb

The SeqFullComb variant library was generated from the DNA of four 7GACT_P cutters called BrA to D (BrA and BrB are the same variants as those given in Table 4), whose sequence is given in Table 4 below. To build SeqFullComb, which contains mutations at positions 32, 33 and 38, two separate overlapping PCR reactions were carried out on each 7GACT_P variant that amplify the 5′ end (aa positions 1-25) or the 3′ end (aa positions 21-167) of the I-CreI derived variants coding sequence. For the 5′ end, PCR amplification is carried out using the Gal10F (SEQ ID NO: 2) and 107Rev (5′-agegatgatgctaccgtcaecgtc-3′, SEQ ID NO: 29). For the 3′ end, PCR amplification is carried out on each of the BrA to BrD variants using the Gal10R (SEQ ID NO: 4) primer and a primer covering residues 21 to 41 specific of the chosen variant sequence. The primers corresponding to the BrA to BrD variants are respectively: SeqBrAFor (5′-ggtagcatcatcgetactattactccaaaccagnvknvkaagtttaaacatnvkctaagettg-3′, SEQ ID NO: 30), SeqBrBFor (5′-ggtageatcatcgettgtatttetccaaaccagnvknvkaagtttaaacatnyketaagettg-3′, SEQ ID NO: 31), SeqBrCFor (5′-ggtagcatcategctgctattaatccaaaccagnyknvkaagtttaaacatnvkctaagettg-3′, SEQ ID NO: 32) and SeqBrDFor (5′-ggtagcatcatcgctgctattactccaaaccagnyknvkaagtttaaacatnvkctaagettg, SEQ ID NO: 33). The nvk codons at positions 32, 33 and 38 allows the degeneracy at these positions among all the 20 possible amino acids but F, L, M, I and V. Then, the four resulting PCR fragments were mixed in an equimolar ratio to 25 ng final and pooled with 25 ng of the Gal10F-107Rev PCR fragment. This mix was then added to 75 ng of vector DNA (pCLS0542) linearized by digestion with NcoI and EagI that were used to transform the yeast Saccharomyces cerevisiae strain FYC2-6A (MATα, trp1Δ63, leu2Δ1, his3Δ200) using a high efficiency LiAc transformation protocol (Gietz and Woods 2002). An intact coding sequence containing mutations at desired positions is generated by in vivo homologous recombination in yeast. 2232 clones were picked for further experiments. They represent 16.5% of the SeqFullComb protein diversity. Results

The SeqFullComb library was generated from the four BrA to BrD variants (Table 5)

TABLE 5
Variants that were used to build the SeqFullComb library.

| Variant | Sequence | SEQ ID NO: |
|---|---|---|
| BrA | 26T28T42S44R77K | 24 |
| BrB | 26C28S42R44R68S77C | 25 |
| BrC | 26A28N77R | 34 |
| BrD | 26A28T42K44V77H | 35 |

The 2232 clones constituting the SeqFullComb library were screened for cleavage of the FullComb_P DNA target using our yeast screening assay. The primary screening yielded 27 positive clones that resulted after sequencing in 22 unique variant sequences. The secondary screening confirmed the cleavage activity toward the FullComb_P target for the vast majority of the variants (FIG. 14). The sequence of the four strongest variants called FC1 to FC4 that have been circled in FIG. 14 is indicated in table 6.

TABLE 6
Variants that showed the strongest cleavage activity
toward the FullComb_P target.

| Variant | Sequence | SEQ ID NO: |
|---|---|---|
| FC1 | 26C28S32T33C38Y42R44R68S77C | 36 |
| FC2 | 26C28S32T33C38C42R44R68S77C | 37 |
| FC3 | 26C28S32G33C38T42R44R68S77C | 38 |
| FC4 | 26C28S32G33C38H42R44R68S77C | 39 |

Taking advantage of the previous screening of the Ulib7NNNN library, this result demonstrates that I-CreI variants with a modified specificity toward nucleotides ±10 to ±4 of the C1221 target can be engineered in only one combinatorial step.
Arnould, S., P. Chames, et al. (2006). “Engineering of Large Numbers of Highly Specific Homing Endonucleases that Induce Recombination on Novel DNA Targets.” J Mol Biol 355(3): 443-58.
Capecchi (2001) Generating mice with targeted mutations. Nat Med, 7, 1086-90.
Chevalier and Stoddard (2001) Homing endonucleases: structural and functional insight into the catalysts of intron/intein mobility. Nucleic Acids Res, 29, 3757-74.
Choulika, Perrin, Dujon and Nicolas (1995) Induction of homologous recombination in mammalian chromosomes by using the 1-SceI system of Saccharomyces cerevisiae. Mol Cell Biol, 15, 1968-73.
Cohen-Tannoudji, Robine, Choulika, Pnto, El Marjou, Babinet, Louvard and Jaisser (1998) I-SceI-induced gene replacement at a natural locus in embryonic stem cells. Mol Cell Biol, 18, 1444-8.
Donoho, Jasin and Berg (1998) Analysis of gene targeting and intrachromosomal homologous recombination stimulated by genomic double-strand breaks in mouse embryonic stem cells. Mol Cell Biol, 18, 4070-8.
Dujon, Colleaux, Jacquier, Michel and Monteilhet (1986) Mitochondrial introns as mobile genetic elements: the role of intron-encoded proteins. Basic Life Sci, 40, 5-27.
Gietz, R. D. and R. A. Woods (2002). “Transformation of yeast by lithium acetate/single-stranded carrier DNA/polyethylene glycol method.” Methods Enzymol 350: 87-96.
Gouble, Smith, Bruneau, Perez, Guyot, Cabaniols, Leduc, Fiette, Ave, Micheau, Duchateau and Paques (2006) Efficient in toto targeted recombination in mouse liver by meganuclease-induced double-strand break. J Gene Med, 8, 616-22.
Haber (1995) In vivo biochemistry: physical monitoring of recombination induced by site-specific endonucleases. Bioessays, 17, 609-20.
Hinnen, Hicks and Fink (1978) Transformation of yeast. Proc Natl Acad Sci USA, 75, 1929-33.
Perez C, Guyot V, Cabaniols J, Gouble A, Micheaux B, Smith J, Leduc S, Paques F, Duchateau P, (2005) BioTechniques vol. 39, n° 1, pp. 109-115
Posfai, Kolisnychenko, Bereczki and Blattner (1999) Markerless gene replacement in Escherichia coli stimulated by a double-strand break in the chromosome. Nucleic Acids Res, 27, 4409-15.
Puchta, Dujon and Hohn (1996) Two different but related mechanisms are used in plants for the repair of genomic double-strand breaks by homologous recombination. Proc Natl Acad Sci USA, 93, 5055-60.
Rothstein (1983) One-step gene disruption in yeast. Methods Enzymol, 101, 202-11.
Rouet, Smih and Jasin (1994) Introduction of double-strand breaks into the genome of mouse cells by expression of a rare-cutting endonuclease. Mol Cell Biol, 14, 8096-106.
Sargent, R. G., Brenneman, M. A., and Wilson, J. H. (1997) Repair of site-specific double-strand breaks in a mammalian chromosome by homologous and illegitimate recombination. Mol Cell Biol, 17, 267-77.
Siebert and Puchta (2002) Efficient Repair of Genomic Double-Strand Breaks by Homologous Recombination between Directly Repeated Sequences in the Plant Genome. Plant Cell, 14, 1121-31.
Smithies {2001) Forty years with homologous recombination. Nat Med, 7, 1083-6.
Thomas and Capecchi (1987) Site-directed mutagenesis by gene targeting in mouse embryo-derived stem cells. Cell, 51, 503-12.

1. An I-CreI variant, comprising at least two substitutions, and obtained by:
(a) constructing a first series of I-CreI variants comprising at least one substitution in a position selected from the group consisting of 26, 28, and 42;
(b) constructing a second series of I-CreI variants comprising at least one substitution in a position selected from the group consisting of 44, 68, and 77;
(c) selecting, screening, or both selecting and screening, the variants (a) which are able to cleave a mutant I-CreI site wherein nucleotides in positions ±7 to ±6 of the wild type I-CreI site have been replaced with nucleotides which are present in positions ±7 to ±6 of a 7NNNN_P DNA target sequence;
(d) selecting, screening, or both selecting and screening, the variants (b) which are able to cleave a mutant I-CreI site wherein nucleotides in positions ±5 to ±4 of the wild type I-CreI site have been replaced with nucleotides which are present in positions ±5 to ±4 of said 7NNNN_P DNA target sequence; and
(e) combining in a single variant, at least one mutation in positions 26, 28, 42, and 44, 68, 77 of two variants from (c) and (d), to obtain a novel homodimeric I-CreI variant which cleaves a sequence wherein a nucleotide quartet in positions ±7 to ±4 is identical to a nucleotide quartet which is present in positions ±7 to ±4 of said 7NNNN_P DNA target sequence,
wherein the I-CreI variant is capable of cleaving a 7NNNN_P palindromic DNA target sequence (SEQ ID NO: 44) other than the wild type I-CreI DNA target sequence (SEQ ID NO: 40).

2. An I-CreI variant, comprising at least two substitutions, and obtained by:
(a′) constructing I-CreI variants having at least one substitution in a position selected from the group consisting of 26, 28, 42, 44, 68, and 77; and
(b′) selecting, screening, or both selecting and screening, the variants (a′) which are able to cleave a 7NNNN_P palindromic DNA target sequence site wherein nucleotides in positions ±7 to ±4 of the wild type I-CreI site have been replaced with nucleotides which are present in positions ±7 to ±4 of a 7NNNN_P DNA target sequence,
wherein the I-CreI variant is capable of cleaving a 7NNNN_P palindromic DNA target sequence (SEQ ID NO: 44) other than the wild type I-CreI DNA target sequence (SEQ ID NO: 40).

3. The I-CreI variant according to claim 1 obtained by:
(A) selecting variants of (c) comprising at least one substitution in a position selected from the group consisting of 26, 28, and 42, which are able to cleave a I-CreI site wherein nucleotides in positions ±7 to ±6 of the wild type I-CreI site have been replaced with nucleotides which are present in positions ±7 to ±6 of a 10NNNNNNN_P DNA target sequence; or
(A′) selecting 7NNNN cutters of (e) and (b′) comprising at least two substitutions in a position selected from the group consisting of 26, 28, 42, 44, 68, and 77, which are able to cleave a mutant I-CreI site wherein nucleotides in positions ±7 to ±4 of the wild type I-CreI site have been replaced with nucleotides which are present in positions ±7 to ±4 of said 10NNNNNNN_P DNA target sequence; and
(B) constructing a series of I-CreI variants comprising at least one substitution in a position selected from the group consisting of 30, 32, 33, 38, and 40;
(C) selecting, screening, or both screening and selecting, the variants (B) which are able to cleave a mutant I-CreI site wherein nucleotides in positions ±10 to ±8 of the wild type I-CreI site have been replaced with nucleotides which are present in positions ±10 to ±8 of said 10NNNNNNN_P DNA target sequence;
(D) combining in a single variant, at least one mutation in positions 26, 28, 42, 44, 68, 77 and 30, 32, 33, 38, 40 of two variants (A) or (A′), and (C), to obtain a novel homodimeric I-CreI variant which cleaves a sequence wherein a nucleotide septet in positions ±10 to ±4 is identical to a nucleotide septet which is present in positions ±10 to ±4 of said 10NNNNNNN_P DNA target sequence,
wherein the I-CreI variant is capable of cleaving a 10NNNNNNN_P palindromic DNA target sequence other than the wild type I-CreI DNA target sequence (SEQ ID NO: 40).

4. The variant of claim 1, which is a heterodimer, resulting from association of a first and a second monomer having different mutations in positions 26 to 42 and 44 to 77 of I-CreI, said heterodimer being capable of cleaving a non-palindromic DNA target sequence.

5. The variant of claim 4, resulting from association of a first and a second monomer having different mutations in positions 26, 28, 42, 44, 68, 77 of I-CreI, said heterodimer being capable of cleaving a non-palindromic DNA target sequence.

6. The variant of claim 4, obtained by:
(i) constructing a third series of variants comprising at least one additional substitution in at least one of the monomers in said heterodimers; and
(ii) combining said third series variants (i) and screening resulting heterodimers for altered cleavage activity against said DNA target.

7. The variant of claim 4, wherein in (i) said at least one substitution are introduced by site directed mutagenesis in a DNA molecule encoding said third series of variants, and/or by random mutagenesis in a DNA molecule encoding said third series of variants.

8. The variant of claim 4, wherein:
(i) and (ii) are repeated at least two times; and
the heterodimers selected in (i) of each further iteration are selected from heterodimers screened in (ii) of a previous iteration which showed increased cleavage activity against said DNA target.

9. The variant of claim 1, wherein a residue at position 75 of I-CreI is not substituted.

10. The variant of claim 1, comprising a substitution on the entire I-CreI sequence improving binding and/or the cleavage properties of the variant towards said DNA target sequence.

11. The variant of claim 10, wherein the substitution involves replacement of initial amino acids with at least one amino acid selected from the group consisting of A, D, E, F, G, H, I, K, M, N, P, Q, R, S, T, Y, C, W, L and V.

12. The variant of claim 11, which is an obligate heterodimer, wherein a first and a second monomer, respectively, further comprises a D137R mutation and a R51D mutation.

13. The variant of claim 12, wherein the first monomer further comprises K7R, E8R, E61R, K96R and L97F or K7R, E8R, F54W, E61R, K96R and L97F mutations, and the second monomer further comprises K7E, F54G, L58M and K96E or K7E, F54G, K57M and K96E mutations.

14. The variant according to claim 1, wherein said variant consists of a single polypeptide chain comprising two monomers or core domains.

15. The variant of claim 14, comprising a first and second monomer connected by a peptide linker.

16. A polynucleotide fragment encoding the variant of claim 1.

17. An expression vector comprising the polynucleotide fragment of claim 16.

18. A process for genome engineering, the process comprising contacting the variant of claim with a cell.
